SEQUENCE LISTING (35 sequences)

SEQ ID NO: 1 - protein, 5 aa, Artificial Sequence
  CD3 HCDR1
TYAMN

SEQ ID NO: 2 - protein, 19 aa, Artificial Sequence
  CD3 HCDR2
RIRSKYNNYATYYADSVKG

SEQ ID NO: 3 - protein, 14 aa, Artificial Sequence
  CD3 HCDR3
HGNFGNSYVSWFAY

SEQ ID NO: 4 - protein, 14 aa, Artificial Sequence
  CD3 LCDR1
GSSTGAVTTSNYAN

SEQ ID NO: 5 - protein, 7 aa, Artificial Sequence
  CD3 LCDR2
GTNKRAP

SEQ ID NO: 6 - protein, 9 aa, Artificial Sequence
  CD3 LCDR3
ALWYSNLWV

SEQ ID NO: 7 - protein, 125 aa, Artificial Sequence
  CD3 VH
EVQLLESGGGLVQPGGSLRLSCAASGFTFSTYAMNWVRQAPGKGLEWVSRIRSKYNNYATYYADSVKGRFTISRDDSKNTLYLQMNSLRAEDTAVYYCVRHGNFGNSYVSWFAYWGQGTLVTVSS

SEQ ID NO: 8 - protein, 109 aa, Artificial Sequence
  CD3 VL
QAVVTQEPSLTVSPGGTVTLTCGSSTGAVTTSNYANWVQEKPGQAFRGLIGGTNKRAPGTPARFSGSLLGGKAALTLSGAQPEDEAEYYCALWYSNLWVFGGGTKLTVL

SEQ ID NO: 9 - protein, 5 aa, Artificial Sequence
  CEA HCDR1
EFGMN

SEQ ID NO: 10 - protein, 17 aa, Artificial Sequence
  CEA HCDR2
WINTKTGEATYVEEFKG

SEQ ID NO: 11 - protein, 12 aa, Artificial Sequence
  CEA HCDR3
WDFAYYVEAMDY

SEQ ID NO: 12 - protein, 11 aa, Artificial Sequence
  CEA LCDR1
KASAAVGTYVA

SEQ ID NO: 13 - protein, 7 aa, Artificial Sequence
  CEA LCDR2
SASYRKR

SEQ ID NO: 14 - protein, 10 aa, Artificial Sequence
  CEA LCDR3
HQYYTYPLFT

SEQ ID NO: 15 - protein, 121 aa, Artificial Sequence
  CEA VH
QVQLVQSGAEVKKPGASVKVSCKASGYTFTEFGMNWVRQAPGQGLEWMGWINTKTGEATYVEEFKGRVTFTTDTSTSTAYMELRSLRSDDTAVYYCARWDFAYYVEAMDYWGQGTTVTVSS

SEQ ID NO: 16 - protein, 108 aa, Artificial Sequence
  CEA VL
DIQMTQSPSSLSASVGDRVTITCKASAAVGTYVAWYQQKPGKAPKLLIYSASYRKRGVPSRFSGSGSGTDFTLTISSLQPEDFATYYCHQYYTYPLFTFGQGTKLEIK

SEQ ID NO: 17 - protein, 5 aa, Artificial Sequence
  CEA HCDR1
DTYMH

SEQ ID NO: 18 - protein, 17 aa, Artificial Sequence
  CEA HCDR2
RIDPANGNSKYVPKFQG

SEQ ID NO: 19 - protein, 12 aa, Artificial Sequence
  CEA HCDR3
FGYYVSDYAMAY

SEQ ID NO: 20 - protein, 15 aa, Artificial Sequence
  CEA LCDR1
RAGESVDIFGVGFLH

SEQ ID NO: 21 - protein, 7 aa, Artificial Sequence
  CEA LCDR2
RASNRAT

SEQ ID NO: 22 - protein, 9 aa, Artificial Sequence
  CEA LCDR3
QQTNEDPYT

SEQ ID NO: 23 - protein, 121 aa, Artificial Sequence
  CEA VH
QVQLVQSGAEVKKPGSSVKVSCKASGFNIKDTYMHWVRQAPGQGLEWMGRIDPANGNSKYVPKFQGRVTITADTSTSTAYMELSSLRSEDTAVYYCAPFGYYVSDYAMAYWGQGTLVTVSS

SEQ ID NO: 24 - protein, 111 aa, Artificial Sequence
  CEA VL
EIVLTQSPATLSLSPGERATLSCRAGESVDIFGVGFLHWYQQKPGQAPRLLIYRASNRATGIPARFSGSGSGTDFTLTISSLEPEDFAVYYCQQTNEDPYTFGQGTKLEIK

SEQ ID NO: 25 - protein, 215 aa, Artificial Sequence
  CEA CD3 bsAb LC(CEA)
DIQMTQSPSSLSASVGDRVTITCKASAAVGTYVAWYQQKPGKAPKLLIYSASYRKRGVPSRFSGSGSGTDFTLTISSLQPEDFATYYCHQYYTYPLFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 26 - protein, 214 aa, Artificial Sequence
  CEA CD3 bsAb LC(CD3)
QAVVTQEPSLTVSPGGTVTLTCGSSTGAVTTSNYANWVQEKPGQAFRGLIGGTNKRAPGTPARFSGSLLGGKAALTLSGAQPEDEAEYYCALWYSNLWVFGGGTKLTVLSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 27 - protein, 694 aa, Artificial Sequence
  CEA CD3 bsAB HC(CEA-CD3-Fc)
QVQLVQSGAEVKKPGASVKVSCKASGYTFTEFGMNWVRQAPGQGLEWMGWINTKTGEATYVEEFKGRVTFTTDTSTSTAYMELRSLRSDDTAVYYCARWDFAYYVEAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDGGGGSGGGGSEVQLLESGGGLVQPGGSLRLSCAASGFTFSTYAMNWVRQAPGKGLEWVSRIRSKYNNYATYYADSVKGRFTISRDDSKNTLYLQMNSLRAEDTAVYYCVRHGNFGNSYVSWFAYWGQGTLVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 28 - protein, 451 aa, Artificial Sequence
  CEA CD3 bsAB HC(CEA-Fc)
QVQLVQSGAEVKKPGASVKVSCKASGYTFTEFGMNWVRQAPGQGLEWMGWINTKTGEATYVEEFKGRVTFTTDTSTSTAYMELRSLRSDDTAVYYCARWDFAYYVEAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 29 - protein, 232 aa, Artificial Sequence
  CEA CD3 bsAb LC(CD3)
EVQLLESGGGLVQPGGSLRLSCAASGFTFSTYAMNWVRQAPGKGLEWVSRIRSKYNNYATYYADSVKGRFTISRDDSKNTLYLQMNSLRAEDTAVYYCVRHGNFGNSYVSWFAYWGQGTLVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 30 - protein, 449 aa, Artificial Sequence
  CEA CD3 bsAB HC(CEA-Fc)
QVQLVQSGAEVKKPGSSVKVSCKASGFNIKDTYMHWVRQAPGQGLEWMGRIDPANGNSKYVPKFQGRVTITADTSTSTAYMELSSLRSEDTAVYYCAPFGYYVSDYAMAYWGQGTLVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 31 - protein, 674 aa, Artificial Sequence
  CEA CD3 bsAB HC(CEA-CD3-Fc)
QVQLVQSGAEVKKPGSSVKVSCKASGFNIKDTYMHWVRQAPGQGLEWMGRIDPANGNSKYVPKFQGRVTITADTSTSTAYMELSSLRSEDTAVYYCAPFGYYVSDYAMAYWGQGTLVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCDGGGGSGGGGSQAVVTQEPSLTVSPGGTVTLTCGSSTGAVTTSNYANWVQEKPGQAFRGLIGGTNKRAPGTPARFSGSLLGGKAALTLSGAQPEDEAEYYCALWYSNLWVFGGGTKLTVLSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 32 - protein, 218 aa, Artificial Sequence
  CEA CD3 bsAb LC(CEA)
EIVLTQSPATLSLSPGERATLSCRAGESVDIFGVGFLHWYQQKPGQAPRLLIYRASNRATGIPARFSGSGSGTDFTLTISSLEPEDFAVYYCQQTNEDPYTFGQGTKLEIKRTVAAPSVFIFPPSDRKLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 33 - protein, 225 aa, Homo sapiens
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 34 - protein, 207 aa, Homo sapiens
MQSGTHWRVLGLCLLSVGVWGQDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRI

SEQ ID NO: 35 - protein, 198 aa, Macaca fascicularis
MQSGTRWRVLGLCLLSIGVWGQDGNEEMGSITQTPYQVSISGTTVILTCSQHLGSEAQWQHNGKNKEDSGDRLFLPEFSEMEQSGYYVCYPRGSNPEDASHHLYLKARVCENCMEMDVMAVATIVIVDICITLGLLLLVYYWSKNRKAKAKPVTRGAGAGGRQRGQNKERPPPVPNPDYEPIRKGQQDLYSGLNQRRI

(19) United States
(12) Patent Application Publication
(10) Pub. No.: US 2022/0017623 A1
(43) Pub. Date: Jan. 20, 2022

(54) TREATMENT OF CANCER USING A CEA CD3 BISPECIFIC ANTIBODY AND A WNT SIGNALING INHIBITOR

(71) Applicant: Hoffmann-La Roche Inc., Little Falls, NJ (US)

(72) Inventors: Marco GERLINGER, London (GB); Reyes GONZALEZ-EXPOSITO, London (GB); Maria SEMIANNIKOVA, London (GB)

(73) Assignee: Hoffmann-La Roche Inc., Little Falls, NJ (US)

(21) Appl. No.: 17/498,512

(22) Filed: Oct. 11, 2021

Related U.S. Application Data
(63) Continuation of application No. PCT/EP2020/060114, filed on Apr. 9, 2020.

(30) Foreign Application Priority Data
Apr. 12, 2019 (EP) 19168811.8

(51) Int. Cl. C07K 16/28; A61K 31/452

(52) U.S. Cl. CPC C07K 16/2827; C07K 2317/31; A61K 31/452; C07K 16/2809

(57) ABSTRACT

The present invention relates to the treatment of cancer, in particular to the treatment of cancer using a CEA CD3 bispecific antibody and a Wnt signaling inhibitor.

### CROSS REFERENCE TO RELATED APPLICATIONS

This application is a continuation of International Patent Application No. PCT/EP2020/060114, filed Apr. 9, 2020, which claims priority to European Application Number 19168811.8 filed Apr. 12, 2019, which are incorporated herein by reference in its entirety.

### SEQUENCE LISTING

This application contains a Sequence Listing which has been submitted electronically in ASCII format and is hereby incorporated by reference in its entirety. Said ASCII copy, created on Oct. 7, 2021, is named P35376-US Sequence listing.txt and is 43,788 bytes in size.

### FIELD OF THE INVENTION

The present invention relates to the treatment of cancer, in particular to the treatment of cancer using a CEA CD3 bispecific antibody and a Wnt signaling inhibitor.

### BACKGROUND

T-cell activating bispecific antibodies are a novel class of cancer therapeutics, designed to engage cytotoxic T cells against tumor cells. The simultaneous binding of such an antibody to CD3 on T-cells and to an antigen expressed on the tumor cells will force a temporary interaction between tumor cell and T cell, causing activation of the T-cell and subsequent lysis of the tumor cell.

The T cell bispecific antibody cibisatamab (RG7802, RO6958688, CEA-TCB) is a novel T-cell activating bispecific antibody targeting carcinoembryonic antigen (CEA) on tumor cells and CD3 on T-cells, that redirects T cells independently of their T cell receptor specificity to tumor cells expressing the CEA glycoprotein at the cell surface (Bacac et al., Oncoimmunology. 2016; 5(8):1-30). A major advantage of T cell redirecting bispecific antibodies is that they mediate cancer cell recognition by T cells independently of neoantigen load. CEA is overexpressed on the cell surface of many colorectal cancers (CRC) and cibisatamab is hence a promising immunotherapy agent for non-hypermutated microsatellite stable (MSS) CRCs.

Cibisatamab has a single binding site for the CD3 epsilon chain on T cells and two CEA binding sites which tune the binding avidity to cancer cells with moderate to high CEA cell surface expression (Bacac et al., Clin Cancer Res. 2016; 22(13):3286-97). This avoids targeting of healthy epithelial cells with low CEA expression levels, which are physiologically present in some tissues. Binding of cibisatamab to CEA on the surface of cancer cells and of CD3 on T cells triggers T cell activation, cytokine secretion and cytotoxic granule release. The phase I trial of cibisatamab in patients with CEA expressing metastatic CRCs that had failed at least two prior chemotherapy regimens showed antitumor activity with radiological shrinkage in 11% (4/36) and 50% (5/10) of patients treated with monotherapy or in combination with PD-L1-inhibiting antibodies, respectively (Argilés et al., Ann Oncol. 2017 Jun. 1; 28(suppl_3):mdx302.003-mdx302.003; Tabemero et al., J Clin Oncol. 2017 May 20; 35(15_suppl):3002). Based on these results, CEA is one of the most promising target antigens for immunotherapy in MSS CRCs. Although some patients in this dose escalation trial were treated with a dose below the final recommended dose, the response rates nevertheless indicate that a subgroup of tumors is resistant to treatment.

It would thus be desirable to increase response rates to and/or therapeutic efficacy of cibisatamab and other CEA-targeted immunotherapy agents, particularly CEA CD3 bispecific antibodies.

### DESCRIPTION OF THE INVENTION

Molecular mechanisms of cibisatamab activity have been investigated in CRC cell lines in vitro using killing assays with peripheral blood mononuclear cells (Bacac et al., Clin Cancer Res. 2016; 22(13):3286-97). This identified CEA expression as a major determinant of cibisatamab sensitivity as only cell lines expressing moderate to high CEA levels were susceptible to T cell mediated killing.

Using patient derived colorectal cancer organoids (PDOs), the present inventors have found that CEA-expression on cancer cells may be increased by treatment with Wnt signaling inhibitors, and thus response rates to and/or therapeutic efficacy of CEA CD3 bispecific antibodies such as cibisatamab may be increased by combining them with Wnt signaling inhibitors.

Accordingly, in a first aspect, the present invention provides a CEA CD3 bispecific antibody for use in the treatment of a cancer in an individual, wherein the treatment comprises administration of the CEA CD3 bispecific antibody in combination with a Wnt signaling inhibitor.

In a further aspect, the invention provides the use of a CEA CD3 bispecific antibody in the manufacture of a medicament for the treatment of cancer in an individual, wherein the treatment comprises administration of the CEA CD3 bispecific antibody in combination with a Wnt signaling inhibitor.

In still a further aspect, the invention provides a method for treating cancer in an individual comprising administering to the individual a CEA CD3 bispecific antibody and a Wnt signaling inhibitor.

In one aspect, the invention also provides a kit comprising a first medicament comprising a CEA CD3 bispecific antibody and a second medicament comprising a Wnt signaling inhibitor, and optionally further comprising a package insert comprising instructions for administration of the first medicament in combination with the second medicament for treating cancer in an individual. The CEA CD3 bispecific antibodies, methods, uses or kits described above and herein, may incorporate, singly or in combination, any of the features described in the following (unless the context dictates otherwise).

The CEA CD3 bispecific antibody herein is a bispecific antibody that specifically binds to CD3 and to CEA. Particularly useful CEA CD3 bispecific antibodies are described e.g. in PCT publication no. WO 2014/131712 and WO 2017/055389 (each incorporated herein by reference in its entirety).

The term “bispecific” means that the antibody is able to specifically bind to at least two distinct antigenic determinants. Typically, a bispecific antibody comprises two antigen binding sites, each of which is specific for a different antigenic determinant. In certain aspects, the bispecific antibody is capable of simultaneously binding two antigenic determinants, particularly two antigenic determinants expressed on two distinct cells.

As used herein, the term “antigenic determinant” is synonymous with “antigen” and “epitope”, and refers to a site (e.g. a contiguous stretch of amino acids or a conformational configuration made up of different regions of non-contiguous amino acids) on a polypeptide macromolecule to which an antigen binding moiety binds, forming an antigen binding moiety-antigen complex. Useful antigenic determinants can be found, for example, on the surfaces of tumor cells, on the surfaces of virus-infected cells, on the surfaces of other diseased cells, on the surface of immune cells, free in blood serum, and/or in the extracellular matrix (ECM).

As used herein, the term “antigen binding moiety” refers to a polypeptide molecule that specifically binds to an antigenic determinant. In one aspect, an antigen binding moiety is able to direct the entity to which it is attached (e.g. a second antigen binding moiety) to a target site, for example to a specific type of tumor cell bearing the antigenic determinant. In another aspect an antigen binding moiety is able to activate signaling through its target antigen, for example a T cell receptor complex antigen. Antigen binding moieties include antibodies and fragments thereof as further defined herein. Particular antigen binding moieties include an antigen binding domain of an antibody, comprising an antibody heavy chain variable region and an antibody light chain variable region. In certain aspects, the antigen binding moieties may comprise antibody constant regions as further defined herein and known in the art. Useful heavy chain constant regions include any of the five isotypes: α, δ, ε, γ, or μ. Useful light chain constant regions include any of the two isotypes: κ and λ.

By “specific binding” is meant that the binding is selective for the antigen and can be discriminated from unwanted or non-specific interactions. The ability of an antigen binding moiety to bind to a specific antigenic determinant can be measured either through an enzyme-linked immunosorbent assay (ELISA) or other techniques familiar to one of skill in the art, e.g. surface plasmon resonance (SPR) technique (analyzed e.g. on a BIAcore instrument) (Liljeblad et al., Glyco J 17, 323-329 (2000)), and traditional binding assays (Heeley, Endocr Res 28, 217-229 (2002)). In one aspect, the extent of binding of an antigen binding moiety to an unrelated protein is less than about 10% of the binding of the antigen binding moiety to the antigen as measured, e.g., by SPR. In certain aspects, an antigen binding moiety that binds to the antigen, or an antibody comprising that antigen binding moiety, has a dissociation constant (K_{D}) of ≤1 μM, ≤100 nM, ≤10 nM, ≤1 nM, ≤0.1 nM, ≤0.01 nM, or ≤0.001 nM (e.g. 10⁻⁸M or less, e.g. from 10⁻⁸M to 10⁻¹³M, e.g., from 10⁻⁹M to 10⁻¹³ M).

“Affinity” refers to the strength of the sum total of non-covalent interactions between a single binding site of a molecule (e.g., a receptor) and its binding partner (e.g., a ligand). Unless indicated otherwise, as used herein, “binding affinity” refers to intrinsic binding affinity which reflects a 1:1 interaction between members of a binding pair (e.g., an antigen binding moiety and an antigen, or a receptor and its ligand). The affinity of a molecule X for its partner Y can generally be represented by the dissociation constant (K_{D}), which is the ratio of dissociation and association rate constants (k_{off} and kₒₙ, respectively). Thus, equivalent affinities may comprise different rate constants, as long as the ratio of the rate constants remains the same. Affinity can be measured by well established methods known in the art, including those described herein. A particular method for measuring affinity is Surface Plasmon Resonance (SPR).

“CD3” refers to any native CD3 from any vertebrate source, including mammals such as primates (e.g. humans), non-human primates (e.g. cynomolgus monkeys) and rodents (e.g. mice and rats), unless otherwise indicated. The term encompasses “full-length,” unprocessed CD3 as well as any form of CD3 that results from processing in the cell. The term also encompasses naturally occurring variants of CD3, e.g., splice variants or allelic variants. In one aspect, CD3 is human CD3, particularly the epsilon subunit of human CD3 (CD3ε). The amino acid sequence of human CD3ε is shown in UniProt (www.uniprot.org) accession no. P07766 (version 144), or NCBI (www.ncbi.nlm.nih.gov/) RefSeq NP_000724.1. See also SEQ ID NO: 34. The amino acid sequence of cynomolgus [Macaca fascicularis] CD3ε is shown in NCBI GenBank no. BAB71849.1. See also SEQ ID NO: 35.

“Carcinoembryonic antigen” or “CEA” (also known as Carcinoembryonic antigen-related cell adhesion molecule 5 (CEACAM5)) refers to any native CEA from any vertebrate source, including mammals such as primates (e.g. humans), non-human primates (e.g. cynomolgus monkeys) and rodents (e.g. mice and rats), unless otherwise indicated. The term encompasses “full-length,” unprocessed CEA as well as any form of CEA that results from processing in the cell. The term also encompasses naturally occurring variants of CEA, e.g., splice variants or allelic variants. In one aspect, CEA is human CEA. The amino acid sequence of human CEA is shown in UniProt (www.uniprot.org) accession no. P06731, or NCBI (www.ncbi.nlm.nih.gov/) RefSeq NP_004354.2. In one aspect, CEA is cell membrane-bound CEA. In one aspect, CEA is CEA expressed on the surface of a cell, e.g. a cancer cell.

As used herein, the terms “first”, “second” or “third” with respect to Fab molecules etc., are used for convenience of distinguishing when there is more than one of each type of moiety. Use of these terms is not intended to confer a specific order or orientation of the bispecific antibody unless explicitly so stated.

The term “valent” as used herein denotes the presence of a specified number of antigen binding sites in an antibody. As such, the term “monovalent binding to an antigen” denotes the presence of one (and not more than one) antigen binding site specific for the antigen in the antibody.

The term “antibody” herein is used in the broadest sense and encompasses various antibody structures, including but not limited to monoclonal antibodies, polyclonal antibodies, multispecific antibodies (e.g. bispecific antibodies), and antibody fragments so long as they exhibit the desired antigen-binding activity.

The terms “full length antibody,” “intact antibody,” and “whole antibody” are used herein interchangeably to refer to an antibody having a structure substantially similar to a native antibody structure.

An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)₂, diabodies, linear antibodies, single-chain antibody molecules (e.g. scFv), and single-domain antibodies. For a review of certain antibody fragments, see Hudson et al., Nat Med 9, 129-134 (2003). For a review of scFv fragments, see e.g. Plückthun, in The Pharmacology of Monoclonal Antibodies, vol. 113, Rosenburg and Moore eds., Springer-Verlag, New York, pp. 269-315 (1994); see also WO 93/16185; and U.S. Pat. Nos. 5,571,894 and 5,587,458. For discussion of Fab and F(ab′)2 fragments comprising salvage receptor binding epitope residues and having increased in vivo half-life, see U.S. Pat. No. 5,869,046. Diabodies are antibody fragments with two antigen-binding sites that may be bivalent or bispecific. See, for example, EP 404,097; WO 1993/01161; Hudson et al., Nat Med 9, 129-134 (2003); and Hollinger et al., Proc Natl Acad Sci USA 90, 6444-6448 (1993). Triabodies and tetrabodies are also described in Hudson et al., Nat Med 9, 129-134 (2003). Single-domain antibodies are antibody fragments comprising all or a portion of the heavy chain variable domain or all or a portion of the light chain variable domain of an antibody. In certain aspects, a single-domain antibody is a human single-domain antibody (Domantis, Inc., Waltham, Mass.; see e.g. U.S. Pat. No. 6,248,516 B1). Antibody fragments can be made by various techniques, including but not limited to proteolytic digestion of an intact antibody as well as production by recombinant host cells (e.g. E. coli or phage), as described herein.

The term “variable region” or “variable domain” refers to the domain of an antibody heavy or light chain that is involved in binding the antibody to antigen. The variable domains of the heavy chain and light chain (VH and VL, respectively) of a native antibody generally have similar structures, with each domain comprising four conserved framework regions (FRs) and three hypervariable regions (HVRs). See, e.g., Kindt et al., Kuby Immunology, 6^{th} ed., W.H. Freeman and Co., page 91 (2007). A single VH or VL domain may be sufficient to confer antigen-binding specificity. As used herein in connection with variable region sequences, “Kabat numbering” refers to the numbering system set forth by Kabat et al., Sequences of Proteins of Immunological Interest, 5th Ed. Public Health Service, National Institutes of Health, Bethesda, Md. (1991).

As used herein, the amino acid positions of all constant regions and domains of the heavy and light chain are numbered according to the Kabat numbering system described in Kabat, et al., Sequences of Proteins of Immunological Interest, 5th ed., Public Health Service, National Institutes of Health, Bethesda, Md. (1991), referred to as “numbering according to Kabat” or “Kabat numbering” herein. Specifically the Kabat numbering system (see pages 647-660 of Kabat, et al., Sequences of Proteins of Immunological Interest, 5th ed., Public Health Service, National Institutes of Health, Bethesda, Md. (1991)) is used for the light chain constant domain CL of kappa and lambda isotype and the Kabat EU index numbering system (see pages 661-723) is used for the heavy chain constant domains (CH1, Hinge, CH2 and CH3), which is herein further clarified by referring to “numbering according to Kabat EU index” in this case.

The term “hypervariable region” or “HVR”, as used herein, refers to each of the regions of an antibody variable domain which are hypervariable in sequence and which determine antigen binding specificity, for example “complementarity determining regions” (“CDRs”). Generally, antibodies comprise six CDRs; three in the VH (HCDR1, HCDR2, HCDR3), and three in the VL (LCDR1, LCDR2, LCDR3). Exemplary CDRs herein include:
(a) hypervariable loops occurring at amino acid residues 26-32 (L1), 50-52 (L2), 91-96 (L3), 26-32 (H1), 53-55 (H2), and 96-101 (H3) (Chothia and Lesk, J. Mol. Biol. 196:901-917 (1987));
(b) CDRs occurring at amino acid residues 24-34 (L1), 50-56 (L2), 89-97 (L3), 31-35b (H1), 50-65 (H2), and 95-102 (H3) (Kabat et al., Sequences of Proteins of Immunological Interest, 5th Ed. Public Health Service, National Institutes of Health, Bethesda, Md. (1991)); and
(c) antigen contacts occurring at amino acid residues 27c-36 (L1), 46-55 (L2), 89-96 (L3), 30-35b (H1), 47-58 (H2), and 93-101 (H3) (MacCallum et al. J. Mol. Biol. 262: 732-745 (1996)).

Unless otherwise indicated, the CDRs are determined according to Kabat et al., supra. One of skill in the art will understand that the CDR designations can also be determined according to Chothia, supra, McCallum, supra, or any other scientifically accepted nomenclature system.

“Framework” or “FR” refers to variable domain residues other than hypervariable region (HVR) residues. The FR of a variable domain generally consists of four FR domains: FR1, FR2, FR3, and FR4. Accordingly, the HVR and FR sequences generally appear in the following order in VH (or VL): FR1-H1(L1)-FR2-H2(L2)-FR3-H3(L3)-FR4.

The “class” of an antibody or immunoglobulin refers to the type of constant domain or constant region possessed by its heavy chain. There are five major classes of antibodies: IgA, IgD, IgE, IgG, and IgM, and several of these may be further divided into subclasses (isotypes), e.g., IgG₁, IgG₂, IgG₃, IgG₄, IgA₁, and IgA₂. The heavy chain constant domains that correspond to the different classes of immunoglobulins are called α, δ, ε, γ, and μ, respectively.

A “Fab molecule” refers to a protein consisting of the VH and CH1 domain of the heavy chain (the “Fab heavy chain”) and the VL and CL domain of the light chain (the “Fab light chain”) of an immunoglobulin.

By a “crossover” Fab molecule (also termed “Crossfab”) is meant a Fab molecule wherein the variable domains or the constant domains of the Fab heavy and light chain are exchanged (i.e. replaced by each other), i.e. the crossover Fab molecule comprises a peptide chain composed of the light chain variable domain VL and the heavy chain constant domain 1 CH1 (VL-CH1, in N- to C-terminal direction), and a peptide chain composed of the heavy chain variable domain VH and the light chain constant domain CL (VH-CL, in N- to C-terminal direction). For clarity, in a crossover Fab molecule wherein the variable domains of the Fab light chain and the Fab heavy chain are exchanged, the peptide chain comprising the heavy chain constant domain 1 CH1 is referred to herein as the “heavy chain” of the (crossover) Fab molecule. Conversely, in a crossover Fab molecule wherein the constant domains of the Fab light chain and the Fab heavy chain are exchanged, the peptide chain comprising the heavy chain variable domain VH is referred to herein as the “heavy chain” of the (crossover) Fab molecule.

In contrast thereto, by a “conventional” Fab molecule is meant a Fab molecule in its natural format, i.e. comprising a heavy chain composed of the heavy chain variable and constant domains (VH-CH1, in N- to C-terminal direction), and a light chain composed of the light chain variable and constant domains (VL-CL, in N- to C-terminal direction).

The term “immunoglobulin molecule” refers to a protein having the structure of a naturally occurring antibody. For example, immunoglobulins of the IgG class are heterotetrameric glycoproteins of about 150,000 daltons, composed of two light chains and two heavy chains that are disulfide-bonded. From N- to C-terminus, each heavy chain has a variable domain (VH), also called a variable heavy domain or a heavy chain variable region, followed by three constant domains (CH1, CH2, and CH3), also called a heavy chain constant region. Similarly, from N- to C-terminus, each light chain has a variable domain (VL), also called a variable light domain or a light chain variable region, followed by a constant light (CL) domain, also called a light chain constant region. The heavy chain of an immunoglobulin may be assigned to one of five types, called α (IgA), δ(IgD), ε (IgE), γ (IgG), or μ (IgM), some of which may be further divided into subtypes, e.g. γ₁ (IgG₁), γ₂ (IgG₂), γ₃ (IgG₃), γ₄ (IgG₄), α₁ (IgA₁) and α₂ (IgA₂). The light chain of an immunoglobulin may be assigned to one of two types, called kappa (κ) and lambda (λ), based on the amino acid sequence of its constant domain. An immunoglobulin essentially consists of two Fab molecules and an Fc domain, linked via the immunoglobulin hinge region.

The term “Fc domain” or “Fc region” herein is used to define a C-terminal region of an immunoglobulin heavy chain that contains at least a portion of the constant region. The term includes native sequence Fc regions and variant Fc regions. Although the boundaries of the Fc region of an IgG heavy chain might vary slightly, the human IgG heavy chain Fc region is usually defined to extend from Cys226, or from Pro230, to the carboxyl-terminus of the heavy chain. However, antibodies produced by host cells may undergo post-translational cleavage of one or more, particularly one or two, amino acids from the C-terminus of the heavy chain. Therefore an antibody produced by a host cell by expression of a specific nucleic acid molecule encoding a full-length heavy chain may include the full-length heavy chain, or it may include a cleaved variant of the full-length heavy chain. This may be the case where the final two C-terminal amino acids of the heavy chain are glycine (G446) and lysine (K447, numbering according to Kabat EU index). Therefore, the C-terminal lysine (Lys447), or the C-terminal glycine (Gly446) and lysine (K447), of the Fc region may or may not be present. Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, Md., 1991 (see also above). A “subunit” of an Fc domain as used herein refers to one of the two polypeptides forming the dimeric Fc domain, i.e. a polypeptide comprising C-terminal constant regions of an immunoglobulin heavy chain, capable of stable self-association. For example, a subunit of an IgG Fc domain comprises an IgG CH2 and an IgG CH3 constant domain.

A “modification promoting the association of the first and the second subunit of the Fc domain” is a manipulation of the peptide backbone or the post-translational modifications of an Fc domain subunit that reduces or prevents the association of a polypeptide comprising the Fc domain subunit with an identical polypeptide to form a homodimer. A modification promoting association as used herein particularly includes separate modifications made to each of the two Fc domain subunits desired to associate (i.e. the first and the second subunit of the Fc domain), wherein the modifications are complementary to each other so as to promote association of the two Fc domain subunits. For example, a modification promoting association may alter the structure or charge of one or both of the Fc domain subunits so as to make their association sterically or electrostatically favorable, respectively. Thus, (hetero)dimerization occurs between a polypeptide comprising the first Fc domain subunit and a polypeptide comprising the second Fc domain subunit, which might be non-identical in the sense that further components fused to each of the subunits (e.g. antigen binding moieties) are not the same. In some aspects the modification promoting association comprises an amino acid mutation in the Fc domain, specifically an amino acid substitution. In a particular aspect, the modification promoting association comprises a separate amino acid mutation, specifically an amino acid substitution, in each of the two subunits of the Fc domain.

The term “effector functions” refers to those biological activities attributable to the Fc region of an antibody, which vary with the antibody isotype. Examples of antibody effector functions include: C1q binding and complement dependent cytotoxicity (CDC), Fc receptor binding, antibody-dependent cell-mediated cytotoxicity (ADCC), antibody-dependent cellular phagocytosis (ADCP), cytokine secretion, immune complex-mediated antigen uptake by antigen presenting cells, down regulation of cell surface receptors (e.g. B cell receptor), and B cell activation.

“Percent (%) amino acid sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, Clustal W, Megalign (DNASTAR) software or the FASTA program package. Those skilled in the art can determine appropriate parameters for aligning sequences, including any algorithms needed to achieve maximal alignment over the full length of the sequences being compared. For purposes herein, however, % amino acid sequence identity values are generated using the ggsearch program of the FASTA package version 36.3.8c or later with a BLOSUM50 comparison matrix. The FASTA program package was authored by W. R. Pearson and D. J. Lipman (1988), “Improved Tools for Biological Sequence Analysis”, PNAS 85:2444-2448; W. R. Pearson (1996) “Effective protein sequence comparison” Meth. Enzymol. 266:227-258; and Pearson et. al. (1997) Genomics 46:24-36, and is publicly available from http://fasta.bioch.virginia.edu/fasta_www2/fasta_down.shtml. Alternatively, a public server accessible at http://fasta.bioch.virginia.edu/fasta_www2/index.cgi can be used to compare the sequences, using the ggsearch (global protein:protein) program and default options (BLOSUM50; open: −10; ext: −2; Ktup=2) to ensure a global, rather than local, alignment is performed. Percent amino acid identity is given in the output alignment header.

An “activating Fc receptor” is an Fc receptor that following engagement by an Fc domain of an antibody elicits signaling events that stimulate the receptor-bearing cell to perform effector functions. Human activating Fc receptors include FcγRIIIa (CD16a), FcγRI (CD64), FcγRIIa (CD32), and FcαRI (CD89).

“Reduced binding”, for example reduced binding to an Fc receptor, refers to a decrease in affinity for the respective interaction, as measured for example by SPR. For clarity, the term includes also reduction of the affinity to zero (or below the detection limit of the analytic method), i.e. complete abolishment of the interaction. Conversely, “increased binding” refers to an increase in binding affinity for the respective interaction.

By “fused” is meant that the components (e.g. a Fab molecule and an Fc domain subunit) are linked by peptide bonds, either directly or via one or more peptide linkers.

The CEA CD3 bispecific antibody comprises a first antigen binding moiety that specifically binds to CD3, and a second antigen binding moiety that specifically binds to CEA.

In one aspect, the first antigen binding moiety comprises a heavy chain variable region comprising the heavy chain CDR (HCDR) 1 of SEQ ID NO: 1, the HCDR2 of SEQ ID NO: 2, and the HCDR3 of SEQ ID NO: 3; and a light chain variable region comprising the light chain CDR (LCDR) 1 of SEQ ID NO: 4, the LCDR2 of SEQ ID NO: 5 and the LCDR3 of SEQ ID NO: 6.

In one aspect, the second antigen binding moiety comprises a heavy chain variable region comprising the heavy chain CDR (HCDR) 1 of SEQ ID NO: 9, the HCDR2 of SEQ ID NO: 10, and the HCDR3 of SEQ ID NO: 11; and a light chain variable region comprising the light chain CDR (LCDR) 1 of SEQ ID NO: 12, the LCDR2 of SEQ ID NO: 13 and the LCDR3 of SEQ ID NO: 14; or (ii) a heavy chain variable region comprising the heavy chain CDR (HCDR) 1 of SEQ ID NO: 17, the HCDR2 of SEQ ID NO: 18, and the HCDR3 of SEQ ID NO: 19; and a light chain variable region comprising the light chain CDR (LCDR) 1 of SEQ ID NO: 20, the LCDR2 of SEQ ID NO: 21 and the LCDR3 of SEQ ID NO: 22.

In a particular aspect, the CEA CD3 bispecific antibody comprises

(i) a first antigen binding moiety that specifically binds to CD3 and comprises a heavy chain variable region comprising the heavy chain CDR (HCDR) 1 of SEQ ID NO: 1, the HCDR2 of SEQ ID NO: 2, and the HCDR3 of SEQ ID NO: 3; and a light chain variable region comprising the light chain CDR (LCDR) 1 of SEQ ID NO: 4, the LCDR2 of SEQ ID NO: 5 and the LCDR3 of SEQ ID NO: 6; and

(ii) a second antigen binding moiety that specifically binds to CEA and comprises a heavy chain variable region comprising the heavy chain CDR (HCDR) 1 of SEQ ID NO: 9, the HCDR2 of SEQ ID NO: 10, and the HCDR3 of SEQ ID NO: 11; and a light chain variable region comprising the light chain CDR (LCDR) 1 of SEQ ID NO: 12, the LCDR2 of SEQ ID NO: 13 and the LCDR3 of SEQ ID NO: 14; or (ii) a heavy chain variable region comprising the heavy chain CDR (HCDR) 1 of SEQ ID NO: 17, the HCDR2 of SEQ ID NO: 18, and the HCDR3 of SEQ ID NO: 19; and a light chain variable region comprising the light chain CDR (LCDR) 1 of SEQ ID NO: 20, the LCDR2 of SEQ ID NO: 21 and the LCDR3 of SEQ ID NO: 22.

In one aspect, the first antigen binding moiety comprises a heavy chain variable region sequence that is at least about 95%, 96%, 97%, 98%, 99% or 100% identical to the amino acid sequence of SEQ ID NO: 7 and a light chain variable region sequence that is at least about 95%, 96%, 97%, 98%, 99% or 100% identical to the amino acid sequence of SEQ ID NO: 8.

In one aspect, the first antigen binding moiety comprises the heavy chain variable region sequence of SEQ ID NO: 7 and the light chain variable region sequence of SEQ ID NO: 8.

In one aspect, the second antigen binding moiety comprises a heavy chain variable region sequence that is at least about 95%, 96%, 97%, 98%, 99% or 100% identical to the amino acid sequence of SEQ ID NO: 15 and a light chain variable region sequence that is at least about 95%, 96%, 97%, 98%, 99% or 100% identical to the amino acid sequence of SEQ ID NO: 16; or (ii) a heavy chain variable region sequence that is at least about 95%, 96%, 97%, 98%, 99% or 100% identical to the amino acid sequence of SEQ ID NO: 23 and a light chain variable region sequence that is at least about 95%, 96%, 97%, 98%, 99% or 100% identical to the amino acid sequence of SEQ ID NO: 24.

In one aspect, the second antigen binding moiety comprises the heavy chain variable region sequence of SEQ ID NO: 15 and the light chain variable region sequence of SEQ ID NO: 16; or (ii) the heavy chain variable region sequence of SEQ ID NO: 23 and the light chain variable region sequence of SEQ ID NO: 24.

In some aspects, the first and/or the second antigen binding moiety is a Fab molecule. In some aspects, the first antigen binding moiety is a crossover Fab molecule wherein either the variable or the constant regions of the Fab light chain and the Fab heavy chain are exchanged. In such aspects, the second antigen binding moiety preferably is a conventional Fab molecule.

In some aspects wherein the first and the second antigen binding moiety of the bispecific antibody are both Fab molecules, and in one of the antigen binding moieties (particularly the first antigen binding moiety) the variable domains VL and VH of the Fab light chain and the Fab heavy chain are replaced by each other,

i) in the constant domain CL of the first antigen binding moiety the amino acid at position 124 is substituted by a positively charged amino acid (numbering according to Kabat), and wherein in the constant domain CH1 of the first antigen binding moiety the amino acid at position 147 or the amino acid at position 213 is substituted by a negatively charged amino acid (numbering according to Kabat EU index); or

ii) in the constant domain CL of the second antigen binding moiety the amino acid at position 124 is substituted by a positively charged amino acid (numbering according to Kabat), and wherein in the constant domain CH1 of the second antigen binding moiety the amino acid at position 147 or the amino acid at position 213 is substituted by a negatively charged amino acid (numbering according to Kabat EU index).

The bispecific antibody does not comprise both modifications mentioned under i) and ii). The constant domains CL and CH1 of the antigen binding moiety having the VH/VL exchange are not replaced by each other (i.e. remain unexchanged).

In a more specific aspect,

i) in the constant domain CL of the first antigen binding moiety the amino acid at position 124 is substituted independently by lysine (K), arginine (R) or histidine (H) (numbering according to Kabat), and in the constant domain CH1 of the first antigen binding moiety the amino acid at position 147 or the amino acid at position 213 is substituted independently by glutamic acid (E), or aspartic acid (D) (numbering according to Kabat EU index); or

ii) in the constant domain CL of the second antigen binding moiety the amino acid at position 124 is substituted independently by lysine (K), arginine (R) or histidine (H) (numbering according to Kabat), and in the constant domain CH1 of the second antigen binding moiety the amino acid at position 147 or the amino acid at position 213 is substituted independently by glutamic acid (E), or aspartic acid (D) (numbering according to Kabat EU index).

In one such aspect, in the constant domain CL of the second antigen binding moiety the amino acid at position 124 is substituted independently by lysine (K), arginine (R) or histidine (H) (numbering according to Kabat), and in the constant domain CH1 of the second antigen binding moiety the amino acid at position 147 or the amino acid at position 213 is substituted independently by glutamic acid (E), or aspartic acid (D) (numbering according to Kabat EU index).

In a further aspect, in the constant domain CL of the second antigen binding moiety the amino acid at position 124 is substituted independently by lysine (K), arginine (R) or histidine (H) (numbering according to Kabat), and in the constant domain CH1 of the second antigen binding moiety the amino acid at position 147 is substituted independently by glutamic acid (E), or aspartic acid (D) (numbering according to Kabat EU index).

In a particular aspect, in the constant domain CL of the second antigen binding moiety the amino acid at position 124 is substituted independently by lysine (K), arginine (R) or histidine (H) (numbering according to Kabat) and the amino acid at position 123 is substituted independently by lysine (K), arginine (R) or histidine (H) (numbering according to Kabat), and in the constant domain CH1 of the second antigen binding moiety the amino acid at position 147 is substituted independently by glutamic acid (E), or aspartic acid (D) (numbering according to Kabat EU index) and the amino acid at position 213 is substituted independently by glutamic acid (E), or aspartic acid (D) (numbering according to Kabat EU index).

In a more particular aspect, in the constant domain CL of the second antigen binding moiety the amino acid at position 124 is substituted by lysine (K) (numbering according to Kabat) and the amino acid at position 123 is substituted by lysine (K) (numbering according to Kabat), and in the constant domain CH1 of the second antigen binding moiety the amino acid at position 147 is substituted by glutamic acid (E) (numbering according to Kabat EU index) and the amino acid at position 213 is substituted by glutamic acid (E) (numbering according to Kabat EU index).

In an even more particular aspect, in the constant domain CL of the second antigen binding moiety the amino acid at position 124 is substituted by lysine (K) (numbering according to Kabat) and the amino acid at position 123 is substituted by arginine (R) (numbering according to Kabat), and in the constant domain CH1 of the second antigen binding moiety the amino acid at position 147 is substituted by glutamic acid (E) (numbering according to Kabat EU index) and the amino acid at position 213 is substituted by glutamic acid (E) (numbering according to Kabat EU index).

In particular aspects, if amino acid substitutions according to the above aspects are made in the constant domain CL and the constant domain CH1 of the second antigen binding moiety, the constant domain CL of the second antigen binding moiety is of kappa isotype.

In some aspects, the first and the second antigen binding moiety are fused to each other, optionally via a peptide linker.

In some aspects, the first and the second antigen binding moiety are each a Fab molecule and either (i) the second antigen binding moiety is fused at the C-terminus of the Fab heavy chain to the N-terminus of the Fab heavy chain of the first antigen binding moiety, or (ii) the first antigen binding moiety is fused at the C-terminus of the Fab heavy chain to the N-terminus of the Fab heavy chain of the second antigen binding moiety.

In some aspects, the CEA CD3 bispecific antibody provides monovalent binding to CD3.

In particular aspects, the CEA CD3 bispecific antibody comprises a single antigen binding moiety that specifically binds to CD3, and two antigen binding moieties that specifically bind to CEA. Thus, in some aspects, the CEA CD3 bispecific antibody comprises a third antigen binding moiety that specifically binds to CEA. In some aspects, the third antigen moiety is identical to the first antigen binding moiety (e.g. is also a Fab molecule and comprises the same amino acid sequences).

In particular aspects, the CEA CD3 bispecific antibody further comprises an Fc domain composed of a first and a second subunit. In one aspect, the Fc domain is an IgG Fc domain. In a particular aspect, the Fc domain is an IgG₁ Fc domain. In another aspect the Fc domain is an IgG₄ Fc domain.

In a more specific aspect, the Fc domain is an IgG₄ Fc domain comprising an amino acid substitution at position S228 (Kabat EU index numbering), particularly the amino acid substitution S228P. This amino acid substitution reduces in vivo Fab arm exchange of IgG₄ antibodies (see Stubenrauch et al., Drug Metabolism and Disposition 38, 84-91 (2010)). In a further particular aspect, the Fc domain is a human Fc domain. In a particularly preferred aspect, the Fc domain is a human IgG₁ Fc domain. An exemplary sequence of a human IgG₁ Fc region is given in SEQ ID NO: 33.

In some aspects wherein the first, the second and, where present, the third antigen binding moiety are each a Fab molecule, (a) either (i) the second antigen binding moiety is fused at the C-terminus of the Fab heavy chain to the N-terminus of the Fab heavy chain of the first antigen binding moiety and the first antigen binding moiety is fused at the C-terminus of the Fab heavy chain to the N-terminus of the first subunit of the Fc domain, or (ii) the first antigen binding moiety is fused at the C-terminus of the Fab heavy chain to the N-terminus of the Fab heavy chain of the second antigen binding moiety and the second antigen binding moiety is fused at the C-terminus of the Fab heavy chain to the N-terminus of the first subunit of the Fc domain; and (b) the third antigen binding moiety, where present, is fused at the C-terminus of the Fab heavy chain to the N-terminus of the second subunit of the Fc domain.

In particular aspects, the Fc domain comprises a modification promoting the association of the first and the second subunit of the Fc domain. The site of most extensive protein-protein interaction between the two subunits of a human IgG Fc domain is in the CH3 domain. Thus, in one aspect said modification is in the CH3 domain of the Fc domain.

In a specific aspect said modification promoting the association of the first and the second subunit of the Fc domain is a so-called “knob-into-hole” modification, comprising a “knob” modification in one of the two subunits of the Fc domain and a “hole” modification in the other one of the two subunits of the Fc domain. The knob-into-hole technology is described e.g. in U.S. Pat. Nos. 5,731,168; 7,695,936; Ridgway et al., Prot Eng 9, 617-621 (1996) and Carter, J Immunol Meth 248, 7-15 (2001). Generally, the method involves introducing a protuberance (“knob”) at the interface of a first polypeptide and a corresponding cavity (“hole”) in the interface of a second polypeptide, such that the protuberance can be positioned in the cavity so as to promote heterodimer formation and hinder homodimer formation. Protuberances are constructed by replacing small amino acid side chains from the interface of the first polypeptide with larger side chains (e.g. tyrosine or tryptophan). Compensatory cavities of identical or similar size to the protuberances are created in the interface of the second polypeptide by replacing large amino acid side chains with smaller ones (e.g. alanine or threonine).

Accordingly, in some aspects, an amino acid residue in the CH3 domain of the first subunit of the Fc domain is replaced with an amino acid residue having a larger side chain volume, thereby generating a protuberance within the CH3 domain of the first subunit which is positionable in a cavity within the CH3 domain of the second subunit, and an amino acid residue in the CH3 domain of the second subunit of the Fc domain is replaced with an amino acid residue having a smaller side chain volume, thereby generating a cavity within the CH3 domain of the second subunit within which the protuberance within the CH3 domain of the first subunit is positionable. Preferably said amino acid residue having a larger side chain volume is selected from the group consisting of arginine (R), phenylalanine (F), tyrosine (Y), and tryptophan (W). Preferably said amino acid residue having a smaller side chain volume is selected from the group consisting of alanine (A), serine (S), threonine (T), and valine (V). The protuberance and cavity can be made by altering the nucleic acid encoding the polypeptides, e.g. by site-specific mutagenesis, or by peptide synthesis.

In a specific such aspect, in the first subunit of the Fc domain the threonine residue at position 366 is replaced with a tryptophan residue (T366W), and in the second subunit of the Fc domain the tyrosine residue at position 407 is replaced with a valine residue (Y407V) and optionally the threonine residue at position 366 is replaced with a serine residue (T366S) and the leucine residue at position 368 is replaced with an alanine residue (L368A) (numbering according to Kabat EU index). In a further aspect, in the first subunit of the Fc domain additionally the serine residue at position 354 is replaced with a cysteine residue (S354C) or the glutamic acid residue at position 356 is replaced with a cysteine residue (E356C) (particularly the serine residue at position 354 is replaced with a cysteine residue), and in the second subunit of the Fc domain additionally the tyrosine residue at position 349 is replaced by a cysteine residue (Y349C) (numbering according to Kabat EU index). In a preferred aspect, the first subunit of the Fc domain comprises the amino acid substitutions S354C and T366W, and the second subunit of the Fc domain comprises the amino acid substitutions Y349C, T366S, L368A and Y407V (numbering according to Kabat EU index).

In some aspects, the Fc domain comprises one or more amino acid substitution that reduces binding to an Fc receptor and/or effector function.

In a particular aspect the Fc receptor is an Fcγ receptor. In one aspect the Fc receptor is a human Fc receptor. In one aspect the Fc receptor is an activating Fc receptor. In a specific aspect the Fc receptor is an activating human Fcγ receptor, more specifically human FcγRIIIa, FcγRI or FcγRIIa, most specifically human FcγRIIIa. In one aspect the effector function is one or more selected from the group of complement dependent cytotoxicity (CDC), antibody-dependent cell-mediated cytotoxicity (ADCC), antibody-dependent cellular phagocytosis (ADCP), and cytokine secretion. In a particular aspect, the effector function is ADCC.

Typically, the same one or more amino acid substitution is present in each of the two subunits of the Fc domain. In one aspect, the one or more amino acid substitution reduces the binding affinity of the Fc domain to an Fc receptor. In one aspect, the one or more amino acid substitution reduces the binding affinity of the Fc domain to an Fc receptor by at least 2-fold, at least 5-fold, or at least 10-fold.

In one aspect, the Fc domain comprises an amino acid substitution at a position selected from the group of E233, L234, L235, N297, P331 and P329 (numberings according to Kabat EU index). In a more specific aspect, the Fc domain comprises an amino acid substitution at a position selected from the group of L234, L235 and P329 (numberings according to Kabat EU index). In some aspects, the Fc domain comprises the amino acid substitutions L234A and L235A (numberings according to Kabat EU index). In one such aspect, the Fc domain is an IgG₁ Fc domain, particularly a human IgG₁ Fc domain. In one aspect, the Fc domain comprises an amino acid substitution at position P329. In a more specific aspect, the amino acid substitution is P329A or P329G, particularly P329G (numberings according to Kabat EU index). In one aspect, the Fc domain comprises an amino acid substitution at position P329 and a further amino acid substitution at a position selected from E233, L234, L235, N297 and P331 (numberings according to Kabat EU index). In a more specific aspect, the further amino acid substitution is E233P, L234A, L235A, L235E, N297A, N297D or P331S. In particular aspects, the Fc domain comprises amino acid substitutions at positions P329, L234 and L235 (numberings according to Kabat EU index). In more particular aspects, the Fc domain comprises the amino acid mutations L234A, L235A and P329G (“P329G LALA”, “PGLALA” or “LALAPG”). Specifically, in preferred aspects, each subunit of the Fc domain comprises the amino acid substitutions L234A, L235A and P329G (Kabat EU index numbering), i.e. in each of the first and the second subunit of the Fc domain the leucine residue at position 234 is replaced with an alanine residue (L234A), the leucine residue at position 235 is replaced with an alanine residue (L235A) and the proline residue at position 329 is replaced by a glycine residue (P329G) (numbering according to Kabat EU index). In one such aspect, the Fc domain is an IgG₁ Fc domain, particularly a human IgG₁ Fc domain.

In a preferred aspect, the CEA CD3 bispecific antibody comprises

(i) a first antigen binding moiety that specifically binds to CD3, comprising a heavy chain variable region comprising the heavy chain CDR (HCDR) 1 of SEQ ID NO: 1, the HCDR2 of SEQ ID NO: 2, and the HCDR3 of SEQ ID NO: 3; and a light chain variable region comprising the light chain CDR (LCDR) 1 of SEQ ID NO: 4, the LCDR2 of SEQ ID NO: 5 and the LCDR3 of SEQ ID NO: 6, wherein the first antigen binding moiety is a crossover Fab molecule wherein either the variable or the constant regions, particularly the constant regions, of the Fab light chain and the Fab heavy chain are exchanged;

(ii) a second and a third antigen binding moiety that specifically bind to CEA, comprising a heavy chain variable region comprising the heavy chain CDR (HCDR) 1 of SEQ ID NO: 9, the HCDR2 of SEQ ID NO: 10, and the HCDR3 of SEQ ID NO: 11; and a light chain variable region comprising the light chain CDR (LCDR) 1 of SEQ ID NO: 12, the LCDR2 of SEQ ID NO: 13 and the LCDR3 of SEQ ID NO: 14, wherein the second and third antigen binding moiety are each a Fab molecule, particularly a conventional Fab molecule;

(iii) an Fc domain composed of a first and a second subunit, wherein the second antigen binding moiety is fused at the C-terminus of the Fab heavy chain to the N-terminus of the Fab heavy chain of the first antigen binding moiety, and the first antigen binding moiety is fused at the C-terminus of the Fab heavy chain to the N-terminus of the first subunit of the Fc domain, and wherein the third antigen binding moiety is fused at the C-terminus of the Fab heavy chain to the N-terminus of the second subunit of the Fc domain.

In one aspect, the first antigen binding moiety comprises a heavy chain variable region sequence that is at least about 95%, 96%, 97%, 98%, 99% or 100% identical to the amino acid sequence of SEQ ID NO: 7 and a light chain variable region sequence that is at least about 95%, 96%, 97%, 98%, 99% or 100% identical to the amino acid sequence of SEQ ID NO: 8.

In one aspect, the first antigen binding moiety comprises the heavy chain variable region sequence of SEQ ID NO: 7 and the light chain variable region sequence of SEQ ID NO: 8.

In one aspect, the second and third antigen binding moiety comprise a heavy chain variable region sequence that is at least about 95%, 96%, 97%, 98%, 99% or 100% identical to the amino acid sequence of SEQ ID NO: 15 and a light chain variable region sequence that is at least about 95%, 96%, 97%, 98%, 99% or 100% identical to the amino acid sequence of SEQ ID NO: 16.

In one aspect, the second and third antigen binding moieties comprise the heavy chain variable region of SEQ ID NO: 15 and the light chain variable region of SEQ ID NO: 16.

The Fc domain according to the above aspects may incorporate, singly or in combination, all of the features described hereinabove in relation to Fc domains.

In one aspect, the antigen binding moieties and the Fc region are fused to each other by peptide linkers, particularly by peptide linkers as in SEQ ID NO: 27 and SEQ ID NO: 28. In one aspect, the CEA CD3 bispecific antibody comprises a polypeptide (particularly two polypeptides) comprising a sequence that is at least 80%, 85%, 90%, 95%, 96%, 97%, 98%, or 99% identical to the sequence of SEQ ID NO: 25, a polypeptide comprising a sequence that is at least 80%, 85%, 90%, 95%, 96%, 97%, 98%, or 99% identical to the sequence of SEQ ID NO: 26, a polypeptide comprising a sequence that is at least 80%, 85%, 90%, 95%, 96%, 97%, 98%, or 99% identical to the sequence of SEQ ID NO: 27, and a polypeptide comprising a sequence that is at least 80%, 85%, 90%, 95%, 96%, 97%, 98%, or 99% identical to the sequence of SEQ ID NO: 28.

In a particularly preferred aspect, the CEA CD3 bispecific antibody comprises a polypeptide (particularly two polypeptides) comprising the sequence of SEQ ID NO: 25, a polypeptide comprising the sequence of SEQ ID NO: 26, a polypeptide comprising the sequence of SEQ ID NO: 27, and a polypeptide comprising the sequence of SEQ ID NO: 28.

In a particularly preferred aspect, the CEA CD3 bispecific antibody is cibisatamab (WHO Drug Information (International Nonproprietary Names for Pharmaceutical Substances), Recommended INN: List 80, 2018, vol. 32, no. 3, p. 438).

In one aspect, the CEA CD3 bispecific antibody comprises

(i) a first antigen binding moiety that specifically binds to CD3, comprising a heavy chain variable region comprising the heavy chain CDR (HCDR) 1 of SEQ ID NO: 1, the HCDR2 of SEQ ID NO: 2, and the HCDR3 of SEQ ID NO: 3; and a light chain variable region comprising the light chain CDR (LCDR) 1 of SEQ ID NO: 4, the LCDR2 of SEQ ID NO: 5 and the LCDR3 of SEQ ID NO: 6, wherein the first antigen binding moiety is a crossover Fab molecule wherein either the variable or the constant regions, particularly the variable regions, of the Fab light chain and the Fab heavy chain are exchanged;

(ii) a second and a third antigen binding moiety that specifically bind to CEA, comprising a heavy chain variable region comprising the heavy chain CDR (HCDR) 1 of SEQ ID NO: 17, the HCDR2 of SEQ ID NO: 18, and the HCDR3 of SEQ ID NO: 19; and a light chain variable region comprising the light chain CDR (LCDR) 1 of SEQ ID NO: 20, the LCDR2 of SEQ ID NO: 21 and the LCDR3 of SEQ ID NO: 22, wherein the second and third antigen binding moiety are each a Fab molecule, particularly a conventional Fab molecule;

(iii) an Fc domain composed of a first and a second subunit capable of stable association,

wherein the second antigen binding moiety is fused at the C-terminus of the Fab heavy chain to the N-terminus of the Fab heavy chain of the first antigen binding moiety, and the first antigen binding moiety is fused at the C-terminus of the Fab heavy chain to the N-terminus of the first subunit of the Fc domain, and wherein the third antigen binding moiety is fused at the C-terminus of the Fab heavy chain to the N-terminus of the second subunit of the Fc domain.

In one aspect, the first antigen binding moiety comprises a heavy chain variable region sequence that is at least about 95%, 96%, 97%, 98%, 99% or 100% identical to the amino acid sequence of SEQ ID NO: 7 and a light chain variable region sequence that is at least about 95%, 96%, 97%, 98%, 99% or 100% identical to the amino acid sequence of SEQ ID NO: 8.

In one aspect, the first antigen binding moiety comprises the heavy chain variable region sequence of SEQ ID NO: 7 and the light chain variable region sequence of SEQ ID NO: 8.

In one aspect, the second and third antigen binding moiety comprise a heavy chain variable region sequence that is at least about 95%, 96%, 97%, 98%, 99% or 100% identical to the amino acid sequence of SEQ ID NO: 23 and a light chain variable region sequence that is at least about 95%, 96%, 97%, 98%, 99% or 100% identical to the amino acid sequence of SEQ ID NO: 24. In one aspect, the second and third antigen binding moieties comprise the heavy chain variable region of SEQ ID NO: 23 and the light chain variable region of SEQ ID NO: 24.

The Fc domain according to the above aspects may incorporate, singly or in combination, all of the features described hereinabove in relation to Fc domains.

In one aspect, the antigen binding moieties and the Fc region are fused to each other by peptide linkers, particularly by peptide linkers as in SEQ ID NO: 30 and SEQ ID NO: 31.

In one aspect, in the constant domain CL of the second and the third Fab molecule under (ii) the amino acid at position 124 is substituted by lysine (K) (numbering according to Kabat) and the amino acid at position 123 is substituted by lysine (K) or arginine (R), particularly by arginine (R) (numbering according to Kabat), and in the constant domain CH1 of the second and the third Fab molecule under (ii) the amino acid at position 147 is substituted by glutamic acid (E) (numbering according to Kabat EU index) and the amino acid at position 213 is substituted by glutamic acid (E) (numbering according to Kabat EU index).

In one aspect, the bispecific antibody comprises a polypeptide comprising a sequence that is at least 80%, 85%, 90%, 95%, 96%, 97%, 98%, or 99% identical to the sequence of SEQ ID NO: 29, a polypeptide comprising a sequence that is at least 80%, 85%, 90%, 95%, 96%, 97%, 98%, or 99% identical to the sequence of SEQ ID NO: 30, a polypeptide comprising a sequence that is at least 80%, 85%, 90%, 95%, 96%, 97%, 98%, or 99% identical to the sequence of SEQ ID NO: 31, and a polypeptide (particularly two polypeptides) comprising a sequence that is at least 80%, 85%, 90%, 95%, 96%, 97%, 98%, or 99% identical to the sequence of SEQ ID NO: 32.

In one aspect, the bispecific antibody comprises a polypeptide comprising the sequence of SEQ ID NO: 29, a polypeptide comprising the sequence of SEQ ID NO: 30, a polypeptide comprising the sequence of SEQ ID NO: 31, and a polypeptide (particularly two polypeptides) comprising the sequence of SEQ ID NO: 32.

Other CEA CD3 bispecific antibodies as will be known to the skilled practitioner are also contemplated for use in the present invention.

In one aspect, the CEA CD3 bispecific antibody is MEDI565 (AMG211, MT111).

The CEA CD3 bispecific antibody herein is used in combination with a Wnt signaling inhibitor. The term “Wnt signaling inhibitor” refers to a molecule that inhibits signaling through the Wnt pathway, in particular the Wnt/β-catenin pathway, also called the canonical Wnt pathway. The Wnt/β-catenin signaling pathway causes an accumulation of β-catenin in the cytoplasm and its eventual translocation into the nucleus to act as a transcriptional coactivator of transcription factors that belong to the TCF/LEF (T-cell factor/lymphoid enhancing factor) family.

The Wnt/β-catenin pathway has been associated with the development of many tumor types, including colorectal cancers.

It requires Wnt ligand binding to Frizzled (Fz) receptors as well as LRP5/6 co-receptors (low density lipoprotein receptor-related protein 5/6) to initiate intracellular signaling via β-catenin nuclear translocation. β-catenin is a highly unstable protein with a tightly controlled cytoplasmic presence. In the absence of Wnt ligands, cytoplasmic β-catenin is targeted by a so-termed degradation complex. This complex is composed of the tumor suppressor adenomatous polyposis coli (APC), the scaffolding protein AXIN and two kinases CK1α (casein kinase 1α) and GSK-3β (glycogen synthase kinase 3 β). These last two components are able to phosphorylate β-catenin on several serine and threonine residues in its N-terminus. Phosphorylated β-catenin is then recognized by β-Transducin, which is part of an ubiquitin ligase complex, leading to poly-ubiquitination and proteasomal degradation of β-catenin. Wnt ligand binding to Frizzled receptors in association with LRPS/6 induces dishevelled (DVL) phosphorylation, which subsequently recruits Axin thereby deconstructing the degradation complex and achieving β-catenin stabilization and subsequent nuclear translocation. In the nucleus, β-catenin can bind members of the TCF/LEF (T-cell factor/lymphoid enhancer factor) family of transcription factors and recruit the transcriptional Kat3 co-activators p300 and/or CBP (CREB-binding protein) to transcribe Wnt target genes and engender chromatin modifications (Duchartre et al., Critical Reviews in Oncology/Hematology 2016, 99, 141-149, incorporated herein by reference in its entirety).

A Wnt signaling inhibitor may be a molecule that targets one or more protein involved in Wnt signaling and inhibits the activity of the Wnt signaling pathway, for example by inhibiting interaction between such protein and other component(s) of the Wnt signaling pathway, promoting degradation of such protein, or inhibiting function (e.g. enzymatic function) of such protein. Exemplary sites of inhibition include, but are not limited to, the Frizzled receptors, the DVL protein, the β-catenin degradation complex (including, e.g., GSK-3β), nuclear β-catenin, and the enzymes porcupine and tankyrase.

Inhibitors of Wnt signaling are reviewed e.g. in Duchartre et al., Critical Reviews in Oncology/Hematology 2016, 99, 141-149, or Tran et al., Protein Science 2017, 26, 650-661 (incorporated herein by reference in their entirety).

In one aspect, the Wnt signaling inhibitor herein is a Wnt/β-catenin signaling inhibitor. In one aspect, the Wnt signaling inhibitor is an inhibitor of the human Wnt signaling pathway, particularly the human Wnt/β-catenin signaling pathway.

In one aspect, the Wnt signaling inhibitor inhibits the interaction of two or more proteins involved in Wnt/β-catenin signaling. In one aspect, the Wnt signaling inhibitor promotes the degradation of one or more proteins involved in Wnt/β-catenin signaling. In one aspect, the Wnt signaling inhibitor inhibits the function of one or more proteins involved in Wnt/β-catenin signaling. In one aspect the Wnt signaling inhibitor targets (e.g. specifically binds to) a component of the Wnt signaling pathway, particularly the Wnt/β-catenin pathway, selected from the group consisting of Frizzled (Fz), Disheveled (DVL), Porcupine, Tankyrase, glycogen synthase kinase 3 β (GSK-3β).

In one aspect, the Wnt signaling inhibitor is a tankyrase inhibitor. Tankyrase 1 and 2 (TNKS/ARTD5 and TNKS2/ARTD5, respectively) are PARP (poly-ADP-ribose polymerase) proteins which are involved in a range of cellular functions including Wnt signaling. TNKS and TNKS2 normally PARylate two components of the destruction complex, AXIN1 and AXIN2, thereby promoting their ubiquitylation and proteosomal degradation, events which minimize the total amount of active β-catenin. Inhibition of TNKS/TNKS2 minimizes AXIN degradation, stabilizes the destruction complex and suppresses Wnt signaling (Elliott et al., Med Chem Comm. 2015, 6, 1687-1692 (incorporated herein by reference in its entirety).

In a specific aspect, the Wnt signaling inhibitor is a tankyrase inhibitor as described in Elliott et al., Med Chem Comm. 2015, 6, 1687-1692, particularly Compound 21 as described therein. The structure of Compound 21 is shown below, wherein R¹ is Me and R² is CH2-N-(4-NMe₂)-piperidine:

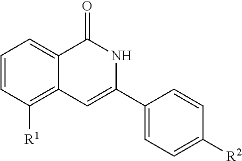

In another specific aspect, the Wnt signaling inhibitor is a tankyrase inhibitor as described in Huang et al., Nature 2009, 461, 614-620 (incorporated herein by reference in its entirety), particularly XAV-939 (CAS no. 284028-89-3).

In another specific aspect, the Wnt signaling inhibitor is a tankyrase inhibitor as described in Chen et al., Nat Chem Biol 2009, 5(2), 100-107 (incorporated herein by reference in its entirety), particularly IWR-1. The structure of IWR-1 is shown below:

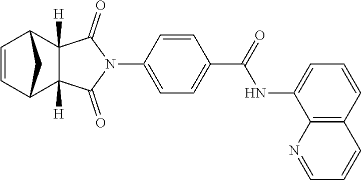 [SMILES: O=C(Nc1cccc2cccnc12)c1ccc(N2C(=O)[C@@H]3[C@H](C2=O)[C@@H]2C=C[C@H]3C2)cc1]

In another specific aspect, the Wnt signaling inhibitor is a tankyrase inhibitor as described in McGonigle et al., Oncotarget 2015, 6, 41307-41323 (incorporated herein by reference in its entirety), particularly E7449 (CAS no. 1140964-99-3).

In another specific aspect, the Wnt signaling inhibitor is a tankyrase inhibitor as described in Waaler et al., Cancer Res 2012, 72, 2822-2832 (incorporated herein by reference in its entirety), particularly JW55 (CAS no. 664993-53-7).

In one aspect, the Wnt signaling inhibitor is a porcupine inhibitor. Porcupine is a member of the membrane-bound O-acetyltransferase (MBOAT) family and is responsible for lipid modification of Wnt and secretion (Duchartre et al., Critical Reviews in Oncology/Hematology 2016, 99, 141-149).

In a specific aspect, the Wnt signaling inhibitor is the porcupine inhibitor LGK974 (CAS no. 1243244-14-5; Liu et al., Proc Natl Acad Sci USA 2013, 110, 20224-20229, incorporated herein by reference in its entirety). The structure of LGK974 is shown below:

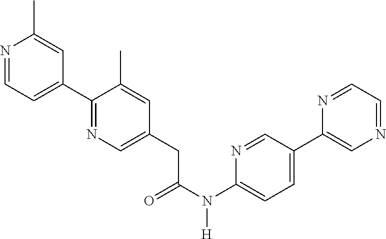 [SMILES: Cc1cc(-c2ncc(CC(=O)Nc3ccc(-c4cnccn4)cn3)cc2C)ccn1]

In another specific aspect, the Wnt signaling inhibitor is a porcupine inhibitor as described in Madan et al., Oncogene 2016, 35, 2197-2207 (incorporated herein by reference in its entirety), particularly ETC-1922159 (ETC-159; CAS no. 1638250-96-0).

In another specific aspect, the Wnt signaling inhibitor is a porcupine inhibitor as described in Madan et al., Kindney Int 2016, 89, 1062-1074 (incorporated herein by reference in its entirety), particularly Wnt-059 (CAS no. 1243243-89-1).

In another specific aspect, the Wnt signaling inhibitor is a porcupine inhibitor as described in Wang et al., J Med Chem 2013, 56, 2700-2704 (incorporated herein by reference in its entirety), particularly IWP-L6 (CAS no. 1427782-89-5) or IWP-2 (CAS no. 686770-61-6).

In one aspect, the Wnt signaling inhibitor is a DVL (disheveled) inhibitor, particularly an inhibitor of the PDZ domain of DVL. The PDZ domain of DVL plays an essential role in DVL-Frizzled receptor interactions and the intracellular transduction of the Wnt signal.

In a specific aspect, the Wnt signaling inhibitor is a DVL inhibitor as described in Shan et al.,

Biochemistry 2005, 44, 15495-15503 (incorporated herein by reference in its entirety), particularly NSC668036 (CAS no. 144678-63-7).

In another specific aspect, the Wnt signaling inhibitor is a DVL inhibitor as described in Grandy et al., J Biol Chem 2009, 284, 16256-16263 (incorporated herein by reference in its entirety), particularly 3289-8625 (CAS no. 294891-81-9).

In a further specific aspect, the Wnt signaling inhibitor is a DVL inhibitor as described in Shan et al., Chem Biol Drug Des 2012, 79, 376-383 (incorporated herein by reference in its entirety), particularly J01-017a.

In another specific aspect, the Wnt signaling inhibitor is a DVL inhibitor as described in Choi et al., Bioorg Med Chem 2016, 24, 3259-3266 (incorporated herein by reference in its entirety), particularly BMD4702 (CAS no. 335206-54-7).

In one aspect, the Wnt signaling inhibitor is a Frizzled inhibitor. Wnt signaling is initiated by the binding of a secreted Wnt molecule to its receptor, Frizzled.

In one aspect, the Wnt signaling inhibitor is an antibody, particularly a monoclonal antibody, that specifically binds to one or more Frizzled receptor. In a specific aspect, the Wnt signaling inhibitor is vantictumab (OMP-18R5).

In one aspect, the Wnt signaling inhibitor comprises the ligand binding domain of a Frizzled receptor. In one aspect, the Wnt signaling inhibitor is a fusion protein comprising the extracellular ligand binding domain of human Frizzled 8 receptor and a human IgG1 Fc domain. In a specific aspect, the Wnt signaling inhibitor is ipafricept (OMP-54F28).

Other Wnt signaling inhibitors as will be known to the skilled practitioner are also contemplated for use in the present invention.

The term “cancer” refers to the physiological condition in mammals that is typically characterized by unregulated cell proliferation. Examples of cancer include but are not limited to, carcinoma, lymphoma, blastoma, sarcoma and leukemia. More particular examples of such cancers include squamous cell cancer, lung cancer (including small-cell lung cancer, non-small cell lung cancer, adenocarcinoma of the lung, non-squamous and squamous carcinoma of the lung), cancer of the peritoneum, hepatocellular cancer, gastric or stomach cancer (including gastrointestinal cancer), pancreatic cancer (including metastic pancreatic cancer), glioblastoma, cervical cancer, ovarian cancer, liver cancer, bladder cancer, hepatoma, breast cancer (including locally advanced, recurrent or metastatic HER-2 negative breast cancer and locally recurrent or metastatic HER2 positive breast cancer), colon cancer, colorectal cancer, endometrial or uterine carcinoma, salivary gland carcinoma, kidney or renal cancer, liver cancer, prostate cancer, vulval cancer, thyroid cancer, hepatic carcinoma and various types of head and neck cancer, as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia); chronic lymphocytic leukemia (CLL); acute lymphoblastic leukemia (ALL); Hairy cell leukemia; chronic myeloblastic leukemia; and post-transplant lymphoproliferative disorder (PTLD), as well as abnormal vascular proliferation associated with phakomatoses, edema (such as that associated with brain tumors), and Meigs' syndrome.

In some aspects of the CEA CD3 bispecific antibodies, methods, uses and kits of the invention, the cancer is a solid tumor cancer. By a “solid tumor cancer” is meant a malignancy that forms a discrete tumor mass (including also tumor metastasis) located at specific location in the patient's body, such as sarcomas or carcinomas (as opposed to e.g. blood cancers such as leukemia, which generally do not form solid tumors). Non-limiting examples of solid tumor cancers include bladder cancer, brain cancer, head and neck cancer, pancreatic cancer, lung cancer, breast cancer, ovarian cancer, uterine cancer, cervical cancer, endometrial cancer, esophageal cancer, colon cancer, colorectal cancer, rectal cancer, gastric cancer, prostate cancer, skin cancer, squamous cell carcinoma, bone cancer, liver cancer and kidney cancer. Other solid tumor cancers that are contemplated in the context of the present invention include, but are not limited to neoplasms located in the: abdomen, bone, breast, digestive system, liver, pancreas, peritoneum, endocrine glands (adrenal, parathyroid, pituitary, testicles, ovary, thymus, thyroid), eye, head and neck, nervous system (central and peripheral), lymphatic system, pelvic, skin, soft tissue, muscles, spleen, thoracic region, and urogenital system. Also included are pre-cancerous conditions or lesions and cancer metastases.

In some aspects, the cancer is a CEA-positive cancer. By “CEA-positive cancer” or “CEA-expressing cancer” is meant a cancer characterized by expression or overexpression of CEA on cancer cells. The expression of CEA may be determined for example by an immunohistochemistry (IHC) or flow cytometric assay. In one aspect, the cancer expresses CEA. In one aspect, the cancer expresses CEA in at least 20%, preferably at least 50% or at least 80% of tumor cells as determined by immunohistochemistry (IHC) using an antibody specific for CEA.

In some aspects, the cancer cells in the patient express PD-L1. The expression of PD-L1 may be determined by an IHC or flow cytometric assay.

In some aspects, the cancer is colon cancer, lung cancer, ovarian cancer, gastric cancer, bladder cancer, pancreatic cancer, endometrial cancer, breast cancer, kidney cancer, esophageal cancer, prostate cancer, or other cancers described herein.

In particular aspects the cancer is a cancer selected from the group consisting of colorectal cancer, lung cancer, pancreatic cancer, breast cancer, and gastric cancer. In a preferred aspect, the cancer is colorectal cancer (CRC). In one aspect, the colorectal cancer is metastatic colorectal cancer (mCRC). In one aspect, the colorectal cancer is microsatellite-stable (MSS) colorectal cancer. In one aspect, the colorectal cancer is microsatellite-stable metastatic colorectal cancer (MSS mCRC).

A “patient”, “subject” or “individual” herein is any single human subject eligible for treatment who is experiencing or has experienced one or more signs, symptoms, or other indicators of cancer. In some aspects, the patient has cancer or has been diagnosed with cancer. In some aspects, the patient has locally advanced or metastatic cancer or has been diagnosed with locally advanced or metastatic cancer. The patient may have been previously treated with a CEA CD3 bispecific antibody or another drug, or not so treated. In particular aspects, the patient has not been previously treated with a CEA CD3 bispecific antibody. The patient may have been treated with a therapy comprising one or more drugs other than a CEA CD3 bispecific antibody before the CEA CD3 bispecific antibody therapy is commenced.

As used herein, “treatment” (and grammatical variations thereof such as “treat” or “treating”) refers to clinical intervention in an attempt to alter the natural course of a disease in the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis.

The CEA CD3 bispecific antibody and the Wnt signaling inhibitor are administered in an effective amount.

An “effective amount” of an agent, e.g. a pharmaceutical composition, refers to an amount effective, at dosages and for periods of time necessary, to achieve the desired therapeutic or prophylactic result.

In one aspect, administration of the CEA CD3 bispecific antibody results in activation of T cells, particularly cytotoxic T cells, particularly at the site of the cancer (e.g. within a solid tumor cancer). Said activation may comprise proliferation of T cells, differentiation of T cells, cytokine secretion by T cells, cytotoxic effector molecule release from T cells, cytotoxic activity of T cells, and expression of activation markers by T cells. In one aspect, the administration of the CEA CD3 bispecific antibody results in an increase of T cell, particularly cytotoxic T cell, numbers at the site of the cancer (e.g. within a solid tumor cancer).

In one aspect, administration of the Wnt signaling inhibitor results in an increase of CEA expression by the cancer. In one aspect, said increase is an increase of CEA expression levels (number of CEA molecules expressed per cell) on the cancer cells. In one aspect, said increase is an increase in the number (or percentage) of cancer cells expressing CEA. The expression of CEA may be determined for example by an immunohistochemistry (IHC) or flow cytometric assay, or by quantification of CEA mRNA (for example by RT-PCR).

In some aspects of the CEA CD3 bispecific antibodies, methods, uses or kits described above and herein, the treatment or administration of the CEA CD3 bispecific antibody and the Wnt inhibitor may result in a response in the individual. In some aspects, the response may be a complete response. In some aspects, the response may be a sustained response after cessation of the treatment. In some aspects, the response may be a complete response that is sustained after cessation of the treatment. In other aspects, the response may be a partial response. In some aspects, the response may be a partial response that is sustained after cessation of the treatment. In some aspects, the response may be improved as compared to treatment or administration of the CEA CD3 bispecific antibody alone (i.e. without the Wnt signaling inhibitor).

In some aspects, the treatment or administration of the CEA CD3 bispecific antibody and the Wnt inhibitor may increase response rates in a patient population, as compared to a corresponding patient population treated with the CEA CD3 bispecific antibody alone (i.e. without the Wnt signaling inhibitor).

The combination therapy of the invention comprises administration of a CEA CD3 bispecific antibody and a Wnt signaling inhibitor.

As used herein, “combination” (and grammatical variations thereof such as “combine” or “combining”) encompasses combinations of a CEA CD3 bispecific antibody and Wnt signaling inhibitor according to the invention wherein the CEA CD3 bispecific antibody and the Wnt signaling inhibitor are in the same or in different containers, in the same or in different pharmaceutical formulations, administered together or separately, administered simultaneously or sequentially, in any order, and administered by the same or by different routes, provided that the CEA CD3 bispecific antibody and the Wnt signaling inhibitor can simultaneously exert their biological effects in the body. For example “combining” CEA CD3 bispecific antibody and a Wnt signaling inhibitor according to the invention may mean first administering the CEA CD3 bispecific antibody in a particular pharmaceutical formulation, followed by administration of the Wnt signaling inhibitor in another pharmaceutical formulation, or vice versa.

The CEA CD3 bispecific antibody and the Wnt signaling inhibitor may be administered in any suitable manner known in the art. In one aspect, the CEA CD3 bispecific antibody and the Wnt signaling inhibitor are administered sequentially (at different times). In another aspect, the CEA CD3 bispecific antibody and the Wnt signaling inhibitor are administered concurrently (at the same time). Without wishing to be bound by theory, it may be advantageous to administer the Wnt signaling inhibitor prior to and/or concurrently with the CEA CD3 bispecific antibody. In some aspects, the CEA CD3 bispecific antibody is in a separate composition as the Wnt signaling inhibitor. In some aspects, the CEA CD3 bispecific antibody is in the same composition as the Wnt signaling inhibitor.

The CEA CD3 bispecific antibody and the Wnt signaling inhibitor can be administered by any suitable route, and may be administered by the same route of administration or by different routes of administration. In some aspects, the CEA CD3 bispecific antibody is administered intravenously, intramuscularly, subcutaneously, topically, orally, transdermally, intraperitoneally, intraorbitally, by implantation, by inhalation, intrathecally, intraventricularly, or intranasally. In a particular aspect, the CEA CD3 bispecific antibody is administered intravenously. In some aspects, the Wnt signaling inhibitor is administered intravenously, intramuscularly, subcutaneously, topically, orally, transdermally, intraperitoneally, intraorbitally, by implantation, by inhalation, intrathecally, intraventricularly, or intranasally. An effective amount of the CEA CD3 bispecific antibody and the Wnt signaling inhibitor may be administered for prevention or treatment of disease. The appropriate route of administration and dosage of the CEA CD3 bispecific antibody and/or the Wnt signaling inhibitor may be determined based on the type of disease to be treated, the type of the CEA CD3 bispecific antibody and the Wnt signaling inhibitor, the severity and course of the disease, the clinical condition of the individual, the individual's clinical history and response to the treatment, and the discretion of the attending physician. Dosing can be by any suitable route, e.g. by injections, such as intravenous or subcutaneous injections, depending in part on whether the administration is brief or chronic. Various dosing schedules including but not limited to single or multiple administrations over various time-points, bolus administration, and pulse infusion are contemplated herein. The CEA CD3 bispecific antibody and the Wnt signaling inhibitor are suitably administered to the patient at one time or over a series of treatments.

Combinations of the invention can be used either alone or together with other agents in a therapy. For instance, a combination of the invention may be co-administered with at least one additional therapeutic agent. In certain aspects, an additional therapeutic agent is an anti-cancer agent, e.g. a chemotherapeutic agent, an inhibitor of tumor cell proliferation, or an activator of tumor cell apoptosis. In particular aspect, the additional therapeutic agent is a PD-L1 binding antagonist, such as atezolizumab.

In some aspects of the CEA CD3 bispecific antibodies, methods, uses or kits described above and herein, the treatment further comprises administration of PD-L1 binding antagonist, particularly atezolizumab.

Combinations of the invention can also be combined with radiation therapy.

A kit as provided herein typically comprises one or more container and a label or package insert on or associated with the container. Suitable containers include, for example, bottles, vials, syringes, IV solution bags, etc. The containers may be formed from a variety of materials such as glass or plastic. The container holds a composition which is by itself or combined with another composition effective for treating, preventing and/or diagnosing the condition and may have a sterile access port (for example the container may be an intravenous solution bag or a vial having a stopper pierceable by a hypodermic injection needle). At least one active agent in the composition is a CEA CD3 bispecific antibody to be used in the combinations of the invention. Another active agent is the Wnt signaling inhibitor to be used in the combinations of the invention, which may be in the same composition and container like the bispecific antibody, or may be provided in a different composition and container. The label or package insert indicates that the composition(s) is/are used for treating the condition of choice, such as cancer.

In one aspect the invention provides a kit intended for the treatment of cancer, comprising in the same or in separate containers (a) a CEA CD3 bispecific antibody, and (b) a Wnt signaling inhibitor, and optionally further comprising (c) a package insert comprising printed instructions directing the use of the combined treatment as a method for treating cancer. Moreover, the kit may comprise (a) a first container with a composition contained therein, wherein the composition comprises a CEA CD3 bispecific antibody; (b) a second container with a composition contained therein, wherein the composition comprises a Wnt signaling inhibitor; and optionally (c) a third container with a composition contained therein, wherein the composition comprises a further cytotoxic or otherwise therapeutic agent. In one aspect, the further therapeutic agent is a PD-L1 binding antagonist, particularly atezolizumab. The kit in these aspects of the invention may further comprise a package insert indicating that the compositions can be used to treat cancer. Alternatively, or additionally, the kit may further comprise a third (or fourth) container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

### BRIEF DESCRIPTION OF THE DRAWINGS

FIG. 1. Growth curves for eight patient derived colorectal cancer organoid (PDO) lines with various cell surface CEA expression levels, that were treated with cibisatamab or an untargeted control antibody during 10 days of co-culture. Each PDO was cultured with T cells from three different allogeneic donors at an effector-to-target (E:T) ratio of 2:1 and means are shown.

FIG. 2. (A) Comparison of the fraction of CEA′ cells in each of eight PDO with the growth reduction achieved with cibisatamab versus the untargeted control antibody at the assay endpoint in FIG. 1. (B) Correlation analysis of growth reduction and the fraction of CEAₕᵢ cells for all PDOs. A linear regression line and the Pearson correlation coefficient and p value of the significance test are shown.

FIG. 3. Cell surface CEA expression of two colorectal cancer organoid lines with and without tankyrase-inhibitor treatment.

FIG. 4. Growth of colorectal cancer organoid lines over 7 days when cultured in the presence of CD8 T cells and cibisatamab or the untargeted control antibody, with or without 2 μM tankyrase-inhibitor (provided either as pre-treatment or continuous treatment).

FIG. 5. Growth of colorectal cancer organoid lines over 7 days when cultured in the presence of CD8 T cells and cibisatamab or the untargeted control antibody, with or without 10 μM tankyrase-inhibitor (provided as pre-treatment).

### EXAMPLES

The following are examples of methods and compositions of the invention. It is understood that various other aspects may be practiced, given the general description provided above.

### Example 1. Cibisatamab Sensitivity of PDOs in an Allogeneic T Cell Co-Culture Assay

Recently developed protocols allow the expansion and long term propagation of cancer cells as so called patient derived organoids (PDOs) from CRC biopsies (Sato et al., Gastroenterology. 2011; 141(5):1762-72). PDOs have been suggested to better represent the biological characteristics of patient tumors than cancer cell lines which were often established decades ago and have undergone changes during long term culture on plastic. The ability to rapidly generate model systems from patients furthermore enables matching of disease stage and prior treatment history to those of patients in whom novel drugs are clinically tested.

In order to assess the sensitivity of patient-derived colorectal cancer organoids (PDOs) to cibisatamab immunotherapy, eight patient derived colorectal cancer organoid lines with various cell surface CEA expression levels were generated and treated with cibisatamab (20 nM) or an untargeted control antibody (20 nM) during 10 days of co-culture with allogeneic CD8 T cells. CD8 T cells were isolated from allogeneic healthy donor peripheral blood mononuclear cells (PBMCs) by magnetic bead sorting and expanded in vitro with IL2 and CD3/CD28 beads for 7-14 days. GFP-tagged CRC PDO cells were then seeded in 96 well plates, T cells were added the following day and the co-cultures were imaged every 2-3 days on an automated 96 well plate fluorescence microscope. Effector to target (E:T) ratios of 2:1 and 5:1 were tested and an E:T of 2:1 was chosen for subsequent experiments as it effectively suppressed growth of the CEAₕᵢ PDO CRC-01 and showed no activity in the presence of the untargeted antibody (DP47-TCB) which was used as a negative control. Co-culture with CD8 T cells without any antibody was included as a further control to enable the identification of alloreactive donor T cells. Co-cultures in which T cells showed alloreactivity (observed in less than one in ten experiments) were excluded from the analysis and assays were repeated until each PDO line was tested with CD8 T cells from 3 independent allogeneic donors.

Eight PDO lines were tested: three CEAₕᵢ PDOs (i.e. containing predominantly CEAₕᵢ cells; CRC-01, CRC-05, CRC-07), four PDOs with mixed CEA expression (i.e. containing large subpopulations of both CEAₕᵢ and CEAₗₒ cells; CRC-02, CRC-03, CRC-04, CRC-08), and one CEAₗₒ PDO (i.e. containing predominantly CEAₗₒ cells; CRC-06).

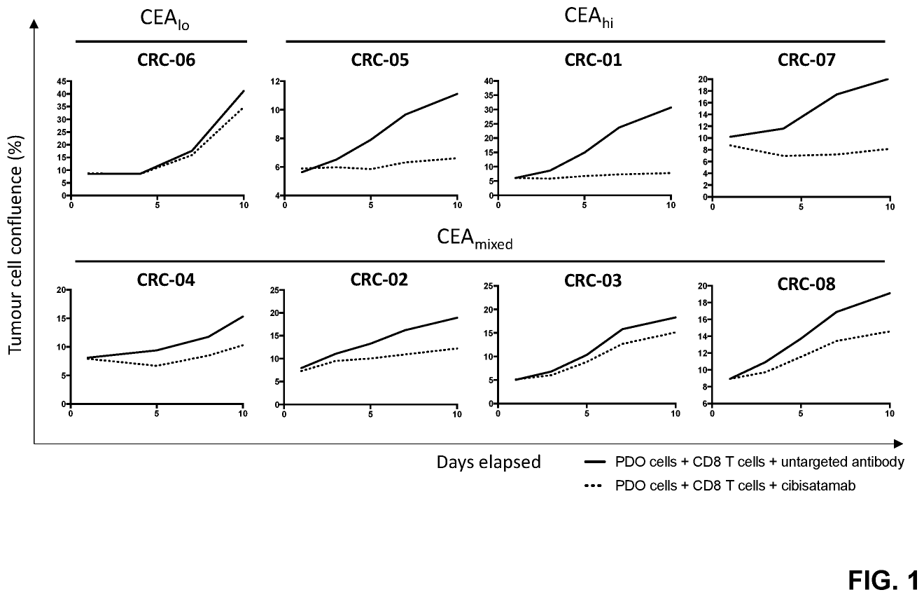

All three CEAₕᵢ PDOs were highly sensitive to treatment with CD8 T cells and cibisatamab whereas the predominantly CEAₗₒ PDO CRC-06 showed resistance under these experimental conditions, as anticipated (FIG. 1). Our assay assessed the impact of cibisatamab over a period of 7-10 days and showed 89-100% growth inhibition in CEAₕᵢ PDOs. This confirms the high efficacy of cibisatamab to re-direct T cells to antigen positive cells in this assay.

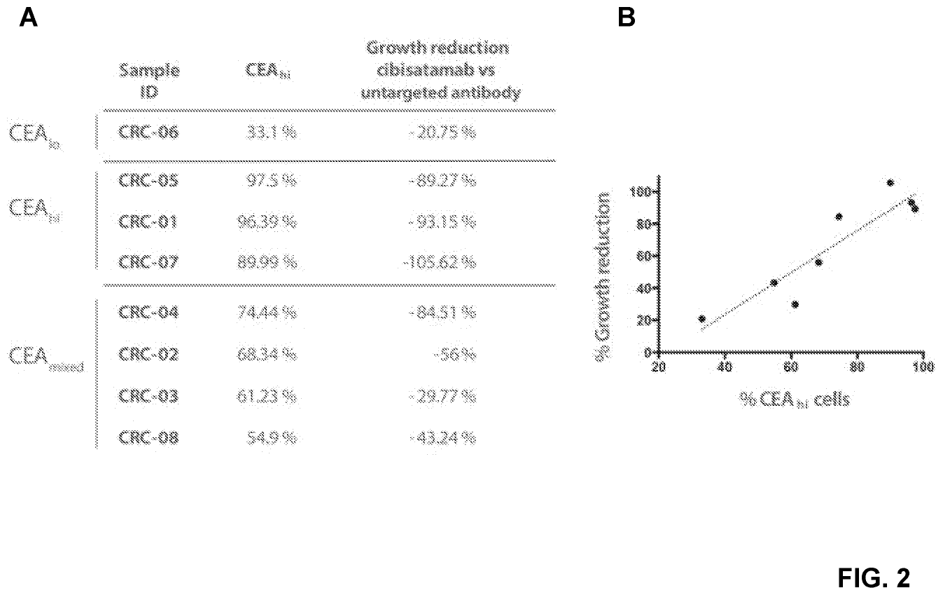

We next tested the four PDOs with mixed CEA expression. Each of these continued to proliferate despite treatment with cibisatamab and T cells, with only a moderate reduction of the cancer cell growth rate compared to controls (FIGS. 1 and 2A). Thus, PDOs with mixed CEA expression only showed a partial response to this CEA targeting immunotherapy. Pearson correlation analysis of the achieved growth reduction with the fraction of CEAₕᵢ cancer cells in each PDO showed a strong and significant correlation of the percentage of cells showing high CEA expression within an organoid line and its sensitivity to cibisatamab (r=0.9152, 95% CI: 0.593 to 0.9848; p=0.0014; FIG. 2B).

This demonstrates that organoids which express uniformly high levels of CEA on the cell surface are sensitive to cibisatamab, organoids with predominantly CEA low cells are resistant and organoids with bimodal/mixed CEA expression only show limited sensitivity.

### Example 2. Cell Surface CEA Expression of Two Colorectal Cancer Organoid Lines with and without Wnt Signalling Inhibitor Treatment

We flow sorted CEAₕᵢ and CEAₗₒ cells from 3 PDOs and performed RNA expression analysis to investigate the mechanisms that regulate CEA expression and generate heterogeneity. We applied gene set enrichment analysis (GSEA) (Subramanian et al., Proc Natl Acad Sci USA. 2005 Sep. 30. 2005 Oct. 25; 102(43):15545-50) to identify potential molecular pathways which associate with CEA gene expression levels. WNT/β-catenin signalling was a significantly enriched signature following multiple testing correction and was upregulated in the CEAₗₒ populations (data not shown). The WNT/β-catenin signalling pathway is genetically activated in the majority of CRCs, most frequently through mutations and loss of heterozygosity of the APC tumor suppressor gene and less commonly through mutations of other regulators of WNT signalling such as RNF43 or in β-catenin/CTNNB1 itself (Network CGA, Nature. 2012 Jul. 18; 487(7407):330-7; Giannakis et al., Nat Genet. 2014 December; 46(12):1264-6). High WNT/β-catenin pathway activity and absence of CEA expression are features of the intestinal crypt bottom where intestinal stem cells reside (Jothy et al., Am J Pathol. 1993 July; 143(1):250-7; Barker et al., Nat Rev Mol Cell Biol. 2013 Dec. 11; 15:19). Moreover, high WNT/β-catenin pathway activity is also a characteristic of colon cancer stem cells (de Sousa et al., Clin Cancer Res. 2011 Feb. 15; 17(4):647 LP-653).

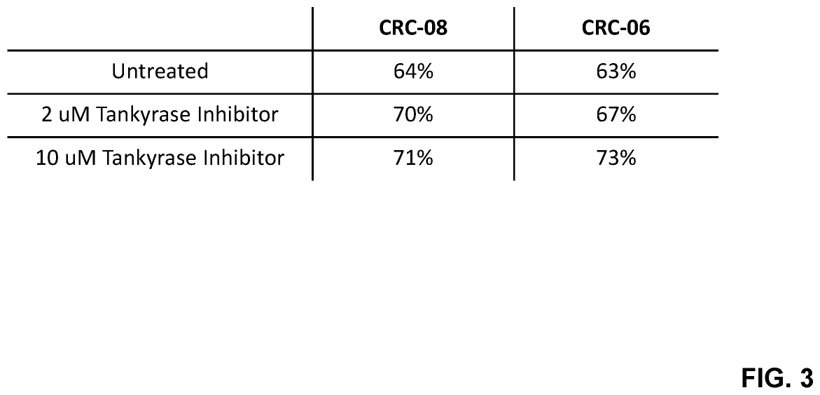

We investigated if pharmacological inhibition of the WNT/β-catenin pathway enhances CEA expression as predicted by these data. Two PDO lines with mixed CEA expression were treated with an inhibitor of WNT signalling: the tankyrase inhibitor compound 21 which inhibits the downstream WNT/β-catenin pathway by stabilizing the β-catenin destruction complex (Elliott et al., Med Chem Comm. 2015; 6(9):1687-92; Mariotti et al., Br J Pharmacol. 2017; 174(24):4611-36). The Wnt signaling inhibitor increased CEA expression and the CEAₕᵢ subpopulation in both PDOs (FIG. 3).

An increase in CEA expression and the CEAₕᵢ subpopulation in PDOs with mixed CEA expression was also seen with another inhibitor of Wnt signaling, the porcupine inhibitor LGK-974 which prevents WNT ligand secretion and hence autocrine and paracrine WNT-receptor activation (results not shown).

These results confirmed a role of WNT/β-catenin signalling as a regulator of CEA expression in CRC PDOs.

### Example 3. Combination Therapy of Cibisatamab and Tankyrase Inhibitor

We investigated growth of two PDO lines with mixed CEA expression (CRC-08, and CRC-06 after prolonged culture as compared to Example 1 above) when treated with the combination of cibisatamab and the tankyrase inhibitor Compound 21.

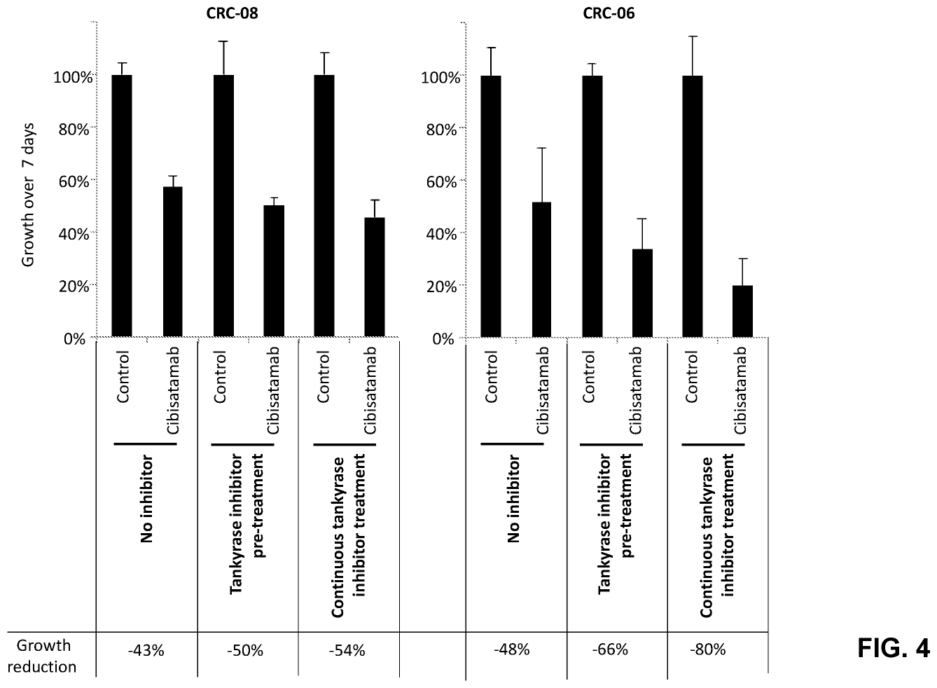

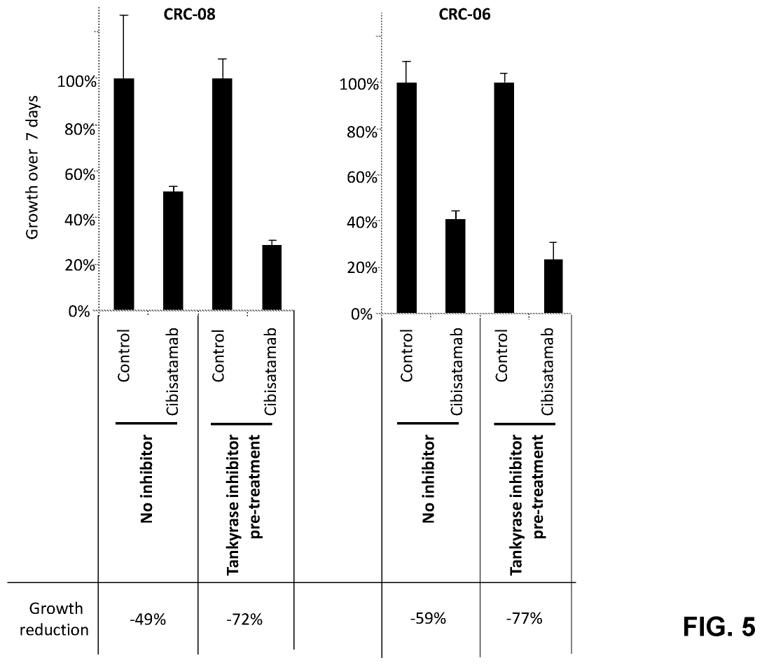

PDOs were cultured over 7 days in the presence of CD8 T cells and 20 nM of cibisatamab or the untargeted control antibody (DP47-TCB). Co-cultures were either performed a) without tankyrase-inhibitor, b) following 48 hours of pre-treatment with tankyrase-inhibitor which was removed at when T cells were added, or c) following 48 hours pre-treatment with tankyrase-inhibitor which was replenished at the time T cells were added for continuous tankyrase-inhibitor exposure. FIG. 4 shows the results for a 2 μM concentration of the tankyrase inhibitor, and FIG. 5 shows the results for a 10 μM concentration of tankyrase-inhibitor (continuous administration of 10 μM tankyrase-inhibitor over the entire assay duration was toxic to cancer cells and the data is not shown). The confluence of the GFP-labelled colorectal cancer organoid cultures was tracked by microscopy for 7 days following addition of T cells and antibody. Growth from the seeding density in the presence of non-targeting control to day 7 was defined as 100%. CD8 T cells had been generated from allogeneic healthy donor cells by extracting peripheral blood mononuclear cells followed by stimulation with IL-2 and CD3-beads and expansion in vitro. Experiments were performed in triplicates and the results shown are averages. Error bars represent one standard deviation. These data demonstrate that tankyrase-inhibitor treatment increases the sensitivity of colorectal cancer spheroid cultures to cibisatamab in a dose- and time-dependent fashion.

### Example 4. Material and Methods

### Human Samples and Cell Lines

Imaging-guided core biopsies from metastatic colorectal cancers that had been treated with at least two prior lines of chemotherapy were obtained from the Prospect C and Prospect R trials (Chief investigator: D. Cunningham, UK national ethics committee approval numbers: 12/L0/0914 and 14/LO/1812, respectively). One endoscopic biopsy from a treatment naïve primary colorectal cancer was obtained from the FOrMAT trial (Chief investigator: N. Starling, UK national ethics committee approval number 13/LO/1274). Trials were run at the Royal Marsden Hospital and all patients provided written informed consent before trial inclusion. Anonymized buffy coats from healthy donors were obtained from the local blood bank (National ethics committee approval number 06/Q1206/106) or through the Improving Outcomes in Cancer biobanking protocol at the Barts Cancer Institute (Chief investigator: T. Powles, UK national ethics committee approval number: 13/EM/0327) from individuals providing written informed consent. DLD-1 and MKN-45 cell lines were obtained from the American Type Culture Collection and were maintained in RPMI 1640 medium supplemented with 10% FBS, 1× Glutamax and 100 units/ml penicillin/streptomicin (Thermo Fisher).

### Generation of Patient Derived Organoids

PDO cultures from CRC-01, CRC-02 and CRC-06 were established directly from core biopsies by rough chopping followed by embedding in growth factor reduced Matrigel (Corning). Very small biopsy fragments were available from CRC-03, CRC-04, CRC-05, CRC-07 and CRC-08 and these were first grafted subcutaneously or under the kidney capsule of female CD1 nude mice by the Tumour Profiling Unit at the Institute of Cancer Research (Home office license number PD498FF8D). Mice were culled once tumors had grown and tumors were removed and dissociated in a gentleMAX Octo dissociator using the Human Tumour Dissociation Kit (Miltenyi Biotec). Mouse cells were magnetically removed using the Mouse Cell Depletion Kit (Miltenyi Biotec), and purified human tumour cells were embedded into growth factor reduced Matrigel. PDOs were expanded in Matrigel as described (Sato et al., Gastroenterology. 2011; 141(5):1762-72) using Advanced DMEM/F12 media supplemented with 1× Glutamax, 100 units/ml penicillin/streptomycin, 1×B27, 1×N2, 10 mM HEPES (all Thermo Fisher), 1 mM N-acetyl cysteine, 10 mM nicotinamide, 10 μM SB202190, 10 nM gastrin, 10 μM Y27632 (Sigma Aldrich), 10 nM prostaglandin E2, 500 nM A-83-01, 100 ng/ml Wnt3a (Biotechne), 50 ng/ml EGF (Merck), 1 μg/ml R-Spondin, 100 ng/ml Noggin, and 100 ng/ml FGF10 (Peprotech). After at least 2 months of continuous growth in the matrigel matrix (minimum of 12 passages), the PDOs were first eGFP tagged (see below) and then adapted to grow in DMEM/F12 (Sigma Aldrich) with 20% fetal bovine serum (FBS), 1× Glutamax, 100 units/ml penicillin/streptomycin containing 2% Matrigel. PDO cultures were maintained in these conditions and used as required for T cell co-culture assays and FACS analysis. Genetic analyses of colon cancer driver genes were performed on each PDO line and these were identical to the mutations that had been identified in the matched tumor biopsies.

Labelling of PDOs with Nuclear eGFP

The nuclei of PDOs were labelled by introducing an eGFP tagged histone 2B construct (pLKO.1-LV-H2B-GFP) (Beronja et al., Nat Med. 2010 July; 16(7):821-7) to enable cell quantification by automated microscopy. For virus generation, HEK-293T cells were cultured in DMEM supplemented with 10% FBS, 1× Glutamax and 100 units/ml penicillin/streptomycin. Lentiviral particles were produced by overnight transfection with a plasmid mixture containing 9 μg of pLKO.1-LV-H2B-GFP, 2.25 pg of psPAX2 packaging plasmid (gift from Didier Trono; Addgene plasmid #12260; http://n2t.net/addgene:12260; RRID: Addgene_12260) and 0.75 ug of pMD2.G envelope plasmid (gift from Didier Trono; Addgene plasmid #12259; http://n2t.net/addgene:12259; RRID: Addgene_12259) using TransIT-293 transfection reagent (Mirus). The cells were media changed the following day, virus harvested after 24 hours and passed through a 0.45 uM filter before use. For lentiviral transduction PDOs were harvested from the cultures in Matrigel and dissociated to single cells using TrypLE Express (Thermo Fisher), and pelleted. The pellets were resuspended in media with the addition of virus and 1 nM polybrene (Sigma Aldrich) and centrifuged at 300 g for 1 hour. The samples were resuspended and plated in culture for between 6 hours and overnight, before replacing the media. Following recovery and expansion, eGFP positive cells were sorted by flow cytometry and further expanded before use.

### Surface CEA Expression Analysis by Flow Cytometry

Cell lines were harvested using enzyme-free Cell Dissociation Buffer (Thermo Fisher) and PDOs with TrypLE Express (Gibco). 2×10⁵ cells were stained with 20 nM of the human anti-human CEA antibody CH1A1A (Roche) and 25 ug/ml of the R-Phycoerythrin conjugated secondary antibody AffiniPure F(ab′)2 Fragment Goat Anti-Human IgG, Fcγ Fragment Specific (Stratech). DRAQ7 (Biostatus) staining was included for dead cell exclusion. CEA expression was analysed on a Sony SH800 flow cytometer. Gate boundaries were set at the trough between high and low CEA populations in PDOs with mixed CEA expression and identical gates were used across all samples. The percentage of CEAₕᵢ and CEAₗₒ populations and mean fluorescence intensities (MFI) were calculated for each PDO.

CD8 T Cells Expansion from Peripheral Blood Mononuclear Cells

Peripheral Blood Mononuclear Cells (PBMCs) were isolated from buffy coats with Ficoll-Paque according to the manufacturer's protocol (GE Healthcare). CD8 T cells were isolated from PBMCs with Human CD8 Dynabeads FlowComp (Thermo Fisher). The purity of CD8 T cells was assessed by flow cytometry (Alexa Fluor 488 anti-human CD8, Sony Biotechnology) and only populations with at least 90% CD8 positive cells were used for expansion with the CD3/CD28 Dynabeads Human T-Activator kit (Thermo Fisher) in RPMI 1640 supplemented with 10% FBS (Biosera), 1× Glutamax, 100 units penicillin/streptomycin and 30 U/mL IL-2 (Sigma Aldrich) following the manufacturer's protocol.

### Co-Culture of PDOs and CD8 T Cells

PDOs were harvested with TrypLE Express and neutralised with DMEM/F12 Ham medium (Sigma Aldrich) with 10% FBS. Cells were filtered through a 70 μm filter, counted and resuspended in phenol-red free RPMI medium (Thermo Fisher) supplemented with 10% FBS (Biosera), 1× Glutamax and 100 units penicillin-streptomycin. On day 0, 5000 tumor cells per well of a 96 well-plate (Corning Special Optics Microplate) were plated. CD8 T cells were added on day 1 at the indicated effector to target (E:T) ratios with 20 nM of cibisatamab or 20 nM of the untargeted negative control antibody DP47-TCB (both provided by Roche). Tumor cells without CD8 T cells and without antibody were also included as controls. All conditions were plated in triplicates and at least 3 different healthy donors were tested on each of the 8 PDOs.

### Cancer Cell Growth Assessment by Immunofluorescence Microscopy

The GFP confluence was quantified every 48h-72h over a 10-day period using the GFP confluence application on the Celigo Imaging Cytometer (Nexcelom Bioscience). GFP confluence analysis was able to track the growth of GFP positive PDO cells over multiple timepoints without erroneously counting the T cells in the co-culture. Confluence analysis was furthermore superior to the counting of cell nuclei which generated inaccurate results in areas of high cancer cell density such as the PDO centre. The main advantage of confluence analysis over measuring spheroid diameters is the ability to track even the growth of PDOs showing highly variable shapes. Growth curves were generated with CD8 T cells from three different healthy blood donors. The percentage growth reduction was calculated from readings taken between days 7 to 9, before PDOs showed growth retardation, likely due to exhaustion of the growth media. In order to calculate the percentage of growth reduction, confluence at day 1 was subtracted and the confluence in wells treated with the DP47-TCB control antibody at the endpoint was set to 100%.

### Wnt/β-Catenin Pathway Inhibition Assay

10⁵ PDO cells/well were seeded in 12 well plates and allowed to attach overnight. Media were changed and cells were treated with DMSO control or with 10 μM tankyrase inhibitor (Compound 21) (Elliott et al., Med Chem Comm. 2015; 6(9):1687-92) or 10 μM porcupine inhibitor (LGK-974, SelleckChem) for 3 days. Cells were harvested using TrypLE Express, stained for CEA with the CH1A1A primary antibody and the R-Phycoerythrin conjugated secondary antibody and analyzed by FACS as described above.

### Statistical Analyses

Pearson correlation analysis and the paired t-tests were performed with the GraphPad Prism software. All p values are two tailed. Gene set enrichment analysis was performed with the GSEA software V3.0 using 5000 gene set permutations and the Hallmarks V6.2 gene set collection (Subramanian et al., Proc Natl Acad Sci USA. 2005 Sep. 30. 2005 Oct. 25; 102(43):15545-50).

Although the foregoing invention has been described in some detail by way of illustration and example for purposes of clarity of understanding, the descriptions and examples should not be construed as limiting the scope of the invention. The disclosures of all patent and scientific literature cited herein are expressly incorporated in their entirety by reference.

1. A method for treating cancer in an individual comprising administering to the individual a CEA CD3 bispecific antibody and a Wnt signaling inhibitor.

2. The method of claim 1, wherein the CEA CD3 bispecific antibody comprises:
(i) a first antigen binding moiety that specifically binds to CD3 and comprises a heavy chain variable region comprising the heavy chain CDR (HCDR) 1 of SEQ ID NO: 1, the HCDR2 of SEQ ID NO: 2, and the HCDR3 of SEQ ID NO: 3; and a light chain variable region comprising the light chain CDR (LCDR) 1 of SEQ ID NO: 4, the LCDR2 of SEQ ID NO: 5 and the LCDR3 of SEQ ID NO: 6; and
(ii) a second antigen binding moiety that specifically binds to CEA and comprises (i) a heavy chain variable region comprising the heavy chain CDR (HCDR) 1 of SEQ ID NO: 9, the HCDR2 of SEQ ID NO: 10, and the HCDR3 of SEQ ID NO: 11; and a light chain variable region comprising the light chain CDR (LCDR) 1 of SEQ ID NO: 12, the LCDR2 of SEQ ID NO: 13 and the LCDR3 of SEQ ID NO: 14; or (ii) a heavy chain variable region comprising the heavy chain CDR (HCDR) 1 of SEQ ID NO: 17, the HCDR2 of SEQ ID NO: 18, and the HCDR3 of SEQ ID NO: 19; and a light chain variable region comprising the light chain CDR (LCDR) 1 of SEQ ID NO: 20, the LCDR2 of SEQ ID NO: 21 and the LCDR3 of SEQ ID NO: 22.

3. The method of claim 2, wherein the CEA CD3 bispecific antibody further comprises a third antigen binding moiety that specifically binds to CEA.

4. The method of claim 2, wherein the CEA CD3 bispecific antibody comprises an Fc domain composed of a first and a second subunit.

5. The method of claim 4, wherein the Fc domain of the CEA CD3 bispecific antibody comprises a modification promoting the association of the first and the second subunit of the Fc domain.

6. The method of claim 4, wherein the Fc domain comprises one or more amino acid substitution that reduces binding to an Fc receptor or effector function.

7. The method of claim 3, wherein the CEA CD3 bispecific antibody comprises
(i) a first antigen binding moiety that specifically binds to CD3, comprising a heavy chain variable region comprising the heavy chain CDR (HCDR) 1 of SEQ ID NO: 1, the HCDR2 of SEQ ID NO: 2, and the HCDR3 of SEQ ID NO: 3; and a light chain variable region comprising the light chain CDR (LCDR) 1 of SEQ ID NO: 4, the LCDR2 of SEQ ID NO: 5 and the LCDR3 of SEQ ID NO: 6, wherein the first antigen binding moiety is a crossover Fab molecule wherein either the variable or the constant regions of the Fab light chain and the Fab heavy chain are exchanged;
(ii) a second and a third antigen binding moiety that specifically bind to CEA, comprising (i) a heavy chain variable region comprising the heavy chain CDR (HCDR) 1 of SEQ ID NO: 9, the HCDR2 of SEQ ID NO: 10, and the HCDR3 of SEQ ID NO: 11; and a light chain variable region comprising the light chain CDR (LCDR) 1 of SEQ ID NO: 12, the LCDR2 of SEQ ID NO: 13 and the LCDR3 of SEQ ID NO: 14; or (ii) a heavy chain variable region comprising the heavy chain CDR (HCDR) 1 of SEQ ID NO: 17, the HCDR2 of SEQ ID NO: 18, and the HCDR3 of SEQ ID NO: 19; and a light chain variable region comprising the light chain CDR (LCDR) 1 of SEQ ID NO: 20, the LCDR2 of SEQ ID NO: 21 and the LCDR3 of SEQ ID NO: 22, wherein the second and third antigen binding moiety are each a Fab molecule;
(iii) an Fc domain composed of a first and a second subunit,
wherein the second antigen binding moiety is fused at the C-terminus of the Fab heavy chain to the N-terminus of the Fab heavy chain of the first antigen binding moiety, and the first antigen binding moiety is fused at the C-terminus of the Fab heavy chain to the N-terminus of the first subunit of the Fc domain, and wherein the third antigen binding moiety is fused at the C-terminus of the Fab heavy chain to the N-terminus of the second subunit of the Fc domain.

8. The method of claim 7, wherein the second and third antigen binding moiety are each a conventional Fab molecule.

9. The method of claim 2, wherein the first antigen binding moiety of the CEA CD3 bispecific antibody comprises a heavy chain variable region sequence that is at least about 95%, 96%, 97%, 98%, 99% or 100% identical to the amino acid sequence of SEQ ID NO: 7 and a light chain variable region sequence that is at least about 95%, 96%, 97%, 98%, 99% or 100% identical to the amino acid sequence of SEQ ID NO: 8, and the second antigen binding moiety of the CEA CD3 bispecific antibody comprises (i) a heavy chain variable region sequence that is at least about 95%, 96%, 97%, 98%, 99% or 100% identical to the amino acid sequence of SEQ ID NO: 15 and a light chain variable region sequence that is at least about 95%, 96%, 97%, 98%, 99% or 100% identical to the amino acid sequence of SEQ ID NO: 16, or (ii) a heavy chain variable region sequence that is at least about 95%, 96%, 97%, 98%, 99% or 100% identical to the amino acid sequence of SEQ ID NO: 23 and a light chain variable region sequence that is at least about 95%, 96%, 97%, 98%, 99% or 100% identical to the amino acid sequence of SEQ ID NO: 24.

10. The method of claim 1, wherein the CEA CD3 bispecific antibody is cibisatamab.

11. The method of claim 1, wherein the Wnt signaling inhibitor is a Wnt/β-catenin signaling inhibitor.

12. The method of claim 1, wherein the Wnt signaling inhibitor targets a component of the Wnt signaling pathway selected from the group consisting of Frizzled (Fz), Disheveled (DVL), Porcupine, tankyrase, and glycogen synthase kinase 3 (3 (GSK-3(3).

13. The method of claim 12, wherein the Wnt signaling inhibitor is a tankyrase inhibitor.

14. The method of claim 13, wherein the Wnt signaling inhibitor is Compound 21: 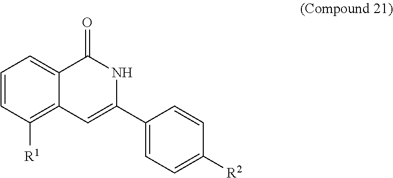
wherein R¹ is Me and R² is CH2-N-(4-NMe₂)-piperidine.

15. The method of claim 1, wherein the treatment further comprises administration of a PD-L1 binding antagonist.

16. The method of claim 15, wherein the PD-L1 antagonist is atezolizumab.

17. The method of claim 1, wherein the cancer is a CEA-positive cancer.

18. The method of claim 17, wherein the cancer is selected from the group consisting of colorectal cancer, lung cancer, pancreatic cancer, breast cancer, and gastric cancer.

19. The method of claim 18, wherein the cancer is colorectal cancer.

20. A kit comprising a first medicament comprising a CEA CD3 bispecific antibody, a second medicament comprising a Wnt signaling inhibitor, and a package insert comprising instructions for administration of the first medicament in combination with the second medicament for treating cancer in an individual.
